(19) United States
(12) Patent Application Publication
(10) Pub. No.: US 2003/0171332 A1
(43) Pub. Date: Sep. 11, 2003

(54) Treatment of respiratory conditions associated with bronchoconstriction with aerosolized hyaluronic acid

(76) Inventors: William M. Abraham, Miami, FL (US); Mario Scuri, Miami, FL (US); Rosanna Forteza, Miami, FL (US); Jing-Wen Kuo, Wakefield, MA (US); Paul Mihalko, Fremont, CA (US); Gregory E. Conner, Miami, FL (US); Matthias Salathe, Coral Gables, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/174,221

(22) Filed: Jun. 17, 2002

Related U.S. Application Data
(63) Continuation-in-part of application No. 09/863,849, filed on May 23, 2001.
(60) Provisional application No. 60/298,369, filed on Jun. 15, 2001.

(51) Int. Cl. A61K 31/737; A61K 31/728; A61K 31/727; A61L 9/04

(52) U.S. Cl. 514/54; 514/56; 424/45

(57) ABSTRACT

A method is disclosed for treating and/or preventing bronchoconstriction induced by neutrophil elastase and tissue kallikrein activity. The method includes administration of aerosolized hyaluronic acid in an amount sufficient to bind to RHAMM (CD168) receptors along the apical surface of the airway epithelium, wherein the hyaluronic acid binds and retains secreted tissue kallikrein, thereby treating and/or preventing bronchoconstriction due to kallikrein activity.

### RELATED APPLICATIONS

This application is a continuation-in-part of pending U.S. application Ser. No. 09/863,849 filed on May 23, 2001, and also claims the benefit of U.S. Provisional Application No. 60/298,369 filed on Jun. 15, 2001 under 35 U.S.C §119(e).

### BACKGROUND OF THE INVENTION

1. Field of the Invention

In a preferred aspect, the present invention relates to formulations and methods for treating respiratory conditions associated with bronchoconstriction and/or airway hyperreactivity. More particularly, the disclosed therapeutic methods involve administering aerosolized hyaluronic acid (“HA”) in amounts sufficient to interact with CD44 and/or receptors for hyaluronic acid-mediated motility (“RHAMM”) disposed on airway epithelium, such that HA binds and thereby inhibits the enzymatic activity of tissue kallikreins (TKs) released in response to a variety of inflammatory stimuli.

2. Description of the Related Art

Respiratory tract disorders are a widespread problem in the United States and throughout the world. Respiratory tract disorders fall into a number of major categories, including inflammatory conditions, infections, cancer, trauma, embolism, and inherited diseases. Lung damage may also be due to physical trauma and exposure to toxins.

Inflammatory conditions of the respiratory tract include asthma, chronic obstructive pulmonary disease, sarcoidosis, and pulmonary fibrosis. Lung infections include pneumonia (bacterial, viral, fungal, or tuberculin) and viral infections. Cancers in the lung may be primary lung cancer, lymphomas, or metastases from other cancerous organs. Trauma to the lung includes lung contusion, barotrauma, and pneumothorax. Embolisms to the lung can consist of air, bacteria, fungi, and blood clots. Inherited lung diseases include cystic fibrosis, and alpha one antitrypsin deficiency. Toxins that can injure the lung include acidic stomach contents (e.g. aspiration pneumonia), inhaled smoke, and inhaled hot air (e.g. from a fire scene).

Patients with any of the above respiratory tract disorders have a component of lung tissue injury. A common contributor to tissue injury in many of these disorders is related to the influx of inflammatory cells, such as neutrophils, macrophages, and eosinophils. Inflammatory cells release noxious enzymes that can damage tissue and trigger physiologic changes. Elastases are one category of noxious enzyme that inflammatory cells release. Elastase enzymes degrade elastic fibers (elastin) in the lung. The damage caused by elastase enzymes may cause the release of tissue kallikrein and may trigger a cascade that attracts additional inflammatory cells to the lung. This influx of additional inflammatory cells release more elastase enzymes, and a “vicious cycle” of lung tissue damage ensues.

Tissue kallikreins (TKs) are a family of serine proteases secreted by salivary glands (Schenkels L C et al. 1995 Crit. Rev. Oral Biol. Med. 6:161-175; Berg T et al. 1990 Acta Physiol. Scand. 139:29-37; Anderson L C et al. 1995 J. Physiol (Lond.) 485:503-51), colon (Berg T et al. 1990 Acta Physiol. Scand. 139:29-37), stomach (Naidoo S et al. 1997 Immunophamacology 36:263-269), uterus (Corthorn J et al. 1997 Biol. Reprod. 56:1432-1438), pituitary gland (Roa J P et al. 1993 Cell Tissue Res. 274:421-427), and pancreas (Bailey G S et al. 1998 Methods Enzymol. 163:115-128) as well as neutrophils, kidney, and endothelial cells (Wu H F et al. 1993 Agents Actions 38:27-31; Geiger R et al. 1981 Methods Enzymol. 80:466-492; Graf K et al. 1994 Eur. J. Clin. Chem. Clin. Biochem. 32:495-500). TK has been identified as the major kininogenase in the airways (Schenkels L C et al. 1995 Crit. Rev. Oral Biol. Med. 6:161-175). It proteolyses both high and low molecular weight kininogen to yield lysyl-bradykinin (kallidin), a potent vasoactive peptide that influences a number of biologic processes including vasodilation, vascular permeability, and bronchoconstriction all of which contribute to the pathophysiology of asthma. TK activity is increased in human nasal and bronchoalveolar lavages (BALF) after antigen challenge (Christiansen S et al. 1992 Am. Rev. Resp. Dis. 145:900-905; Christiansen S et al. 1987 J. Clin. Invest. 79:188-197; Baumgarten C R et al. 1986 J. Immunology 137:1323-1328). Bronchoconstriction and/or airway hyperreactivity caused by a wide range of inflammatory stimuli such as allergen, metabisulfite, ozone, and bacterial supernatant are associated with increased levels of immunoreactive kinins and increased TK activity in BALF of allergic sheep (Abraham W M et al. 1994 Am. J. Resp. Crit. Care Med. 149:A533; Forteza R et al. 1994 Am. J. Resp. Crit. Care Med. 149(4):A158; Forteza R et al. 1994 Am. J. Resp. Crit. Care Med. 149:687-693; Mansour E et al. 1992 J. Appl. Physiol. 72:1831-1837; Forteza R et al. 1996 Am. J. Resp. Crit. Care Med. 154:36-42). Elastase causes bronchoconstriction in sheep via a bradykinin-mediated mechanism (Scuri M et al. 2000 J. Appl. Physiol. 89(4):1397-1402) and also releases TK from ovine tracheal glands (Forteza R et al. 1997 Am. J. Resp. Crit. Care Med. 155(4):A357).

Recently, Forteza et al. (Forteza R et al. 1999 Am. J. Resp. Cell Mol. Biol. 21:666-674) showed that hyaluronic acid (“HA”), also called hyaluronan, binds to TK on the airway surface, thereby reducing its activity. Thus, HA may be effective as a therapeutic agent in respiratory conditions associated with increased TK activity. HA is a large linear polymer with a molecular mass from about 2×10⁵ to about 10×10⁶ daltons formed by a repeating disaccharide structure of glucuronic acid and N-acetylglucosamine. HA is present in all vertebrates and some strains of streptococci (De Angelis P L et al. 1993 J. Biol. Chem. 268:19181-19184) and is abundant in virtually all biologic fluids. Its biological actions include cell-cell and cell-matrix signaling, regulation of cell migration and proliferation as well a providing the fundamental biochemical properties of many tissues (Fraser J R et al. 1997 J. Intern. Med. 242:27-33). In the lung HA accumulates as part of the fibroproliferative response to injury and tissue remodeling. These actions are mediated by the binding to two major cell surface receptors: CD44 and RHAMM (receptor for hyaluronic acid-mediated motility). CD44 binding stimulates signaling via Rac (Oliferenko S et al. 2000 J. Cell Biol. 148:1159-1164; Bourguignon L Y et al. 2000 J. Biol. Chem. 275:1829-1838), and Ras (Fitzgerald K A et al. 2000 J. Immunol. 164:2053-2063). RHAMM is also thought to signal via Ras but, unlike CD44, it is present both on the cell surface and intracellularly (Zhang S. et al. 1998 J. Biol. Chem. 273:11342-11348; Hofmann M et al. 1998 J. Cell. Sci. 111:1673-1684; Fieber C et al. 1999 Gene 226:41-50).

Accordingly there is a need for a treatment wherein inhaled HA is administered in amounts sufficient to inhibit the increases in TK activity resulting from various inflammatory stimuli, thus treating and/or preventing bronchoconstriction and/or airway hyperreactivity.

### SUMMARY OF THE INVENTION

In accordance with one embodiment of the present invention, a method is disclosed for treating or preventing respiratory conditions associated with tissue kallikrein-induced bronchoconstriction and/or airway hyperreactivity. The method comprises administering to a mammal in need thereof an amount of an aerosolized formulation comprising a polysaccharide capable of binding to CD44 and/or RHAMM cell surface receptors at a location along the airway epithelium. The amount of polysaccharide is sufficient to sequester tissue kallikrein to the location along the airway epithelium, wherein the enzymatic activity of the tissue kallikrein is inhibited, thereby treating or preventing the respiratory condition.

Preferably, the polysaccharide is a glycosaminoglycan, selected from the group consisting of hyaluronic acid, chondroitin sulfate A, chondroitin sulfate B, chondroitin sulfate C, heparan sulfate and heparin. Most preferably, the polysaccharide is hyaluronic acid.

In a variation to the method, the aerosolized formulation further comprises a step of preparing a liquid formulation comprising the polysaccharide, wherein the concentration of the polysaccharide is less than about 5 mg/ml and the molecular weight of the polysaccharide is less than about 1.5×10⁶ Daltons. The formulation is then aerosolized to form a breathable mist such that the particle size of the polysaccharide is less than about 10 microns. The formulation is then delivered in an effective amount by inhalation of the breathable mist.

Preferably, the molecular weight of the polysaccharide is less than about 587,000 Daltons. More preferably, the molecular weight of the polysaccharide is less than about 220,000 Daltons, and most preferably, the molecular weight of the polysaccharide is about 150,000 Daltons.

In a preferred aspect of the invention, the breathable mist is formed by a nebulizer. Preferably, the nebulizer is operated at a pressure of at least about 15 psi. Alternatively, the nebulizer operates at a pressure of at least about 30 psi.

In a variation to the present invention, the polysaccharide is chemically modified. The modification may comprise cross-linking. Alternatively, the modification comprises addition of a functional group selected from the group consisting of sulfate group, carboxyl group, lipophilic side chain, acetyl group, and ester.

Preferably, the location along the airway epithelium is a ciliated border of the airway epithelium.

In a preferred embodiment, the amount of polysaccharide is in a range of about 10 μg/kg body weight/day to about 10 mg/kg body weight/day.

In variations to the present invention, the method may further comprise the step of monitoring tissue kallikrein activity via bronchoalveolar lavage or airway resistivity.

In accordance with another embodiment of the present invention, a method is disclosed for treating or preventing respiratory conditions associated with tissue kallikrein-induced bronchoconstriction and/or airway hyperreactivity. The method comprises administering to a mammal in need thereof an aerosolized formulation comprising hyaluronic acid in an amount sufficient to bind to RHAMM cell surface receptors at a ciliated border of an airway epithelium and sequester tissue kallikrein to the ciliated border, thereby treating or preventing the respiratory condition.

In accordance with another embodiment of the present invention, a method is disclosed for preventing acute bronchoconstriction due to an induction of neutrophil elastase. The method comprises administering to a mammal at least four hours prior to the induction an aerosolized formulation comprising hyaluronic acid at a concentration of at least 0.1% (w/v) with an average molecular weight of 150,000 daltons.

In a variation to the method, the hyaluronic acid may be administered at least eight hours prior to the induction at a concentration of at least 0.5% (w/v).

### BRIEF DESCRIPTION OF THE DRAWINGS

FIG. 1 shows the effect of low molecular weight hyaluronic acid (LMW-HA) on elastase-induced bronchoconstriction. Elastase-induced bronchoconstriction was short-lived and reached its peak immediately after challenge to resolve within 30 minutes. LMW-HA 0.2% completely blocked this response whereas LMW-HA 0.1% and 0.05% showed a differential protection indicating a dose-related effect. Values are expressed as mean±SE for 6 sheep. *P<0.001 vs elastase and LMW-HA 0.1 and 0.05%. +P<0.001 vs elastase and LMW-HA 0.2 and 0.05%.

FIG. 2 shows the effect of high molecular weight hyaluronic acid (HMW-HA) on elastase-induced bronchoconstriction. Elastase-induced bronchoconstriction was short-lived and reached its peak immediately after challenge to resolve within 30 minutes. HMW-HA 0.05% completely blocked this response whereas HMW-HA 0.01% showed a partial protection and HMW-HA 0.005% was ineffective against the elastase-induced airway response indicating a dose-related effect. Values are expressed as mean±SE for 6 sheep. *P<0.001 vs elastase and HMW-HA 0.01 and 0.005%. +P<0.001 vs elastase and HMW-HA 0.05 and 0.005%.

FIG. 3 shows the dose-dependent and molecular weight-dependent effect of hyaluronic acid on elastase-induced bronchial response. The percent protection against elastase-induced bronchoconstriction is plotted against different concentrations of either LMW-HA or HMW-HA (on a logarithmic scale). Both molecular weights of HA show a dose-related effect. Furthermore HMW-HA achieved an almost complete degree of protection at a much lower concentration than LMW-HA, indicating a molecular weight-dependent effect. Values in the figure are expressed as mean±SE for 6 sheep.

FIG. 4 shows the effect of HMW-HA on elastase-induced TK activity in sheep BALF. Elastase challenge caused a significant increase in TK activity in sheep BALF. This increase was inhibited by pretreatment with HMW-HA 0.05%, a dose that proved effective in blocking the elastase-induced bronchoconstriction. The lower dose of HMW-HA (0.005%), which didn't affect the elastase-induced airway responses, however couldn't block the elastase-induced increase in sheep BALF TK activity. TK activity is expressed as arbitrary units (1 Unit=change in optical density at 405 nm in 24 h). Values are expressed as mean±SE for 7-8 sheep. *P<0.05 vs control and HA 0.005%.

FIG. 5 shows staining for bronchial tissue kallikrein (TK), airway lactoperoxidase (LPO) and HA in airway epithelial cells (DIC images). Cultured airway epithelial cells are shown in panels A-C. Control cells exposed to pre-immune serum (A) do not show any non-specific labeling. Cells stained for LPO (B) or TK (C) using specific antibodies and DAB revealed specific labeling along cilia. D-I. Visualization in tracheal sections using a biotinylated HA-binding protein and NBT/BCIP reveals that HA is localized to the ciliary border of the epithelium in addition to its known localization in the submucosal interstitium (D). Labeling with anti-LPO antibodies and NBT/BCIP (E) or anti-TK antibodies and DAB (F) reveals specific staining along the ciliary border of the airway epithelium. Incubation with hyaluronidase (37° C. overnight) removes specific staining for HA (G), LPO (H), and TK (I), whereas chondroitinase ABC, used at neutral pH where it does not have hyaluronidase activity, does not remove any specific labeling (not shown). All bars are 101 μM.

FIG. 6 shows immunohistochemistry and immunocytochemistry for RHAMM (CD168) in airway epithelial cells. Labeling for RHAMM using a specific antibody (R36) and NBT/BCIP reveals its presence in the apical portion of ciliated cells including the cilia themselves (A), while pre-immune serum shows no non-specific staining (B). RHAMM is also expressed on the surface of cultured, non-permeabilized airway epithelial cells (C). All bars are 10 μm. Using specific primers for RHAMM (bolded in D) and an ovine airway epithelial cDNA library, PCR reactions yielded a 249 by cDNA fragment (nucleotide sequence shown in D) with a deduced amino-acid sequence that was 91% and 81% identical to the human and the mouse sequence, respectively.

FIG. 7 shows HA-induced CBF increase is blocked by anti-RHAMM antibodies. Tracings show continuous recordings of ciliary beat frequency (“CBF”) in primary cultures of ovine tracheal epithelial cells in response to exogenous HA (50 μg/ml). All cells respond to 20 μM ATP with a statistically indistinguishable transient increase in CBF. (A/B) Cells incubated with a non-specific control IgG (before and during the experiment) respond to HA with an increase in CBF. There are two types of responses: (A) a transient, but continuous increase in CBF, and (B) an oscillatory response. (C) reveals that the CBF response to HA is blocked using a functionally blocking anti-RHAMM antibody.

FIG. 8 shows the effect of HA on TK and albumin movement by the mucociliary transport system. Tracheas from freshly sacrificed sheep were opened at their membranous portion. White arrows point to the proximal end of the trachea and represent a length of 2 cm surface. A/B and C/D show the same trachea at time 0 (A/C) and after 30 minutes (B/D). (A) Fluorescein-labeled TK and rhodamine-labeled albumin were mixed and applied to the tracheal surface, revealing an orange fluorescence at time 0. (B) After 30 minutes of incubation at 37° C. (humidified), fluorescein-labeled TK did not move as indicated by the stripe of green fluorescence at the location of application, whereas albumin, represented by red fluorescence, has separated from TK towards the proximal end of the trachea (movement approx. 2.5 cm in this experiment). (C/D) The shown trachea was pretreated with hyaluronidase as described in methods. Again, the TK/albumin mixture was applied (C), represented by an orange fluorescence. After digestion of HA, both TK and albumin are transported without separation for approx. 2.5 cm during the 30 minute observation period (D).

FIG. 9 shows the effect of 0.1% HA pretreatment on human neutrophil elastase-induced bronchoconstriction in sheep.

FIG. 10 shows the effect of 0.5% HA pretreatment on human neutrophil elastase-induced bronchoconstriction in sheep.

FIG. 11 shows tissue distribution ³H-HA clearance.

FIG. 12 shows the time course of ³H-HA clearance from lung tissue and lavage fluid.

### DETAILED DESCRIPTION OF THE PREFERRED EMBODIMENT

Enzymes such as lactoperoxidase and tissue kallikrein (TK), which are secreted onto epithelial surfaces, play a vital role in innate mucosal defense. In contrast to the belief that their mucosal presence is maintained by secretion, one aspect of the present invention relates to the observation that the enzymes of the airway mucosa bind to surface-associated glycosaminoglycans (GAG's; e.g., HA), providing an apical enzyme pool “ready for use” independent of secretion. It is demonstrated herein that the model airway defense enzymes, lactoperoxidase and TK, bind to HA, which is bound to the epithelial mucosa through interaction with CD44 and/or RHAMM (CD168). The binding of these enzymes to HA resulted in inhibition of TK activity, but not lactoperoxidase activity. HA itself also stimulated ciliary beating by binding to RHAMM (CD168). Thus, it has been shown by the inventors that HA plays a previously unrecognized role in mucosal host defense by retaining and regulating certain enzymes, e.g., TK, important for homeostasis at the apical surface, while simultaneously stimulating ciliary clearance of foreign material.

In a preferred aspect of the present invention, methods and materials are disclosed for the treatment or mitigation of pulmonary disorders associated with increased TK activity along the respiratory epithelium by delivery to the lungs of polysaccharides and/or derivatives thereof, preferably HA. The polysaccharide formulations disclosed herein may be useful in treating and/or preventing a variety of pulmonary conditions and disorders, including for example emphysema, as detailed in U.S. Pat. No. 5,633,003 to Cantor and U.S. patent application Ser. No. 09/079,209; the disclosures of which are incorporated herein in their entirety by reference thereto. In addition, other therapeutic indications for polysaccharide administration to the lung includes: stabilizing the lung matrix (tissue which contains the alveolar sacs and bronchii) by forming a polymer network within the lung matrix; placing a polysaccharide barrier on the matrix fibers of the lung to reduce or eliminate future degradation of the lung fibers, or to protect the fibers from noxious agents while they undergo repair; providing a polysaccharide coating of the lung matrix, surface, bronchioles, and/or alveoli that enhances the moisture content, lubrication, or elastic recoil of the lung; replacing HA in conditions where HA is diminished (e.g. aging, emphysema); providing a bulking agent in the lung to reinforce delicate anatomic structures such as alveolar walls (e.g. blebs); providing a lubricant between the internal & external pleura; providing a viscoelastic agent to facilitate elastic lung recoil; providing a dressing to facilitate healing of injured lung tissue; reducing and/or preventing inflammation due to infection, cancer, irritation, allergy, etc.; treating bronchospasm; lubricating and/or loosening mucous; binding to cell receptors to influence cell activity in the lung, such as ciliary cell beating, cell attachment (or adhesion), or cell migration.

Binding in the context of the present invention includes both covalent and non-covalent binding. The binding may be either high or low affinity. The binding may be temporary such that the binding is a coating sufficient to provide a temporary interation. Examples of binding forces include, but are not limited to, ionic and covalent bonds, hydrogen binding, electrostatic forces, dipole interactions, or Van der Waals forces. Binding can be defined empirically by those skilled in the art by fluorescence microscopy, following—conjugation of the compound with a fluorescent dye, as discussed in greater detail below.

The polysaccharide or carbohydrate moiety may be administered alone or in combination with other polysaccharides or carbohydrate moieties, with or without a suitable carrier. Such suitable carriers include, but are not limited to, carriers like saline solution, DMSO, alcohol, or water. It may be composed of naturally occurring, chemically modified, or artificially synthesized compounds which are wholly or partially composed of polysaccharides or other carbohydrate moieties, and which are capable of binding to elastic fibers.

The amount of the polysaccharide or carbohydrate moiety administered daily may vary from about 1 μg/kg to about 1 mg/kg of body weight, depending on the site and route of administration. More preferably, the dose is in a range of from about 50 μg/kg body weight/day to about 500 μg/kg body weight/day. Most preferably, the dose is in a range of from about 100 μg/kg body weight/day to about 300 μg/kg body weight/day. For example, a 50 minute exposure to an aerosol containing a 0.1% solution of bovine tracheal hyaluronic acid (HA) in water (1 mg/ml) was effective in coating hamster lung elastic fibers with HA.

In one aspect of the present invention, a method for using a formulation comprising a polysaccharide to treat and/or prevent a respiratory disorder. In one aspect, the method comprises the steps of selecting formulation parameters, which include the molecular weight, the concentration and the viscosity of polysaccharide, such that when aerosolized, the formulation yields a droplet size adapted for delivery to the lungs. The formulation is then aerosolized to form an aerosol, and delivered to the lungs.

Another aspect of the invention relates to a method for delivering to the lung alveoli, also referred to as the respiratory zone or deep lung, a polysaccharide or derivative thereof. The method comprises selecting a preparation of the polysaccharide or derivative having a molecular weight sufficient to provide a desired therapeutic profile. Then, preparing a delivery formulation comprising the selected preparation of polysaccharide or derivative at a concentration which when aerosolized yields a particle size suitable for delivery to the deep lung. The delivery formulation is then aerosolized to form an aerosol, and delivered to the deep lung.

In another mode of the method for delivering to a lung alveolus an amount of a formulation comprising a polysaccharide or derivative, formulation parameters are selected. These parameters include molecular weight, concentration and viscosity of the polysaccharide or derivative, such that when aerosolized, the formulation yields a droplet size adapted for delivery to the lung alveoli.

Another aspect of the invention relates to a method of treating and/or preventing respiratory disorders by the use of hyaluronic acid, its derivatives, other polysaccharides, and other polysaccharides, either alone or in conjunction with pharmaceuticals, delivered by nebulization or instillation, etc., to the lung tissues.

Another aspect of the invention relates to a method for delivering to a selected target site in a lung, a polysaccharide or derivative thereof. The method comprises the steps of preparing a formulation comprising the polysaccharide or derivative at a molecular weight and concentration adapted to yield a desired rheological profile for effective mass transfer during aerosolization or nebulization; and selecting a delivery apparatus and operation parameters, such that when aerosolized, the formulation yields a median droplet size of less than 10 microns, preferably less than 5 microns and most preferably between 0.05-5 microns, with the size range of approximately 2-5 microns being adapted for delivery to conducting airways, or the size range of approximately 0.5-2 microns being adapted for delivery to the deep lung or respiratory zone.

Another aspect of the invention relates to a formulation comprising HA, other polysaccharides and derivatives thereof having a molecular weight, a concentration and a viscosity that are selected to provide a desired therapeutic profile, and to be deliverable by aerosolization to the deep lung for the treatment of a respiratory disorder.

Another aspect of the invention relates to a formulation comprising HA conjugated with a second active agent, wherein the formulation has a molecular weight, a concentration and a viscosity that are selected to be deliverable in aerosol form to an alveolus for the treatment of a respiratory disorder.

Another aspect of the invention relates to a formulation comprising a polysaccharide and a second agent, wherein the formulation is adapted to be delivered to a lung and also adapted to provide systemic delivery of the second agent.

The biocompatible polymers useful in the present invention include without limitation, natural and synthetic, native and modified, anionic or acidic saccharides, disaccharides, oligosaccharides, polysaccharides and in particular, the glycosaminoglycans (GAGs) or acid mucopolysaccharides, which include both non-sulfated (e.g., HA and chondroitin) and sulfated forms (e.g., chondroitin sulfate, dermatan sulfate, heparan sulfate, heparin sulfate, and keratan sulfate). This class of acid mucopolysaccharides can be defined more generally as any polysaccharide having a repeating unit of a dissacharide composed of a hexosamine, e.g., N-acetylated glucosamine, and a uronic acid, e.g., D-glucuronic acid, with or without a sulfate group. Also included within the class of polysaccharides in accordance with the present invention are dextrans, lectins, glucans, and mannans. In one variation of the present invention, the formulation may comprise a combination of one or more polysaccharides. In addition, this invention is intended to cover polymer derivatives that may be produced by the addition of various chemical groups, such as hydroxyl, carboxyl, sulfate groups, bonded to the polymer.

In accordance with one aspect of the invention, polysaccharides may be obtained via any variety of methods in the prior art such as bacterial fermentation, via processing from animal or plant tissue, or via chemical synthesis. The formulation of the material will enable delivery of the polysaccharides into the lung via aerosol, dry powder delivery, or direct instillation in such a fashion as to adequately cover target, or susceptible, or diseased tissue. Specifically, the concentration, molecular weight, and viscosity will be such that the material can be dispersed throughout the target site(s) within the lungs, and allow for a desired dosing frequency (e.g., preferably about every six hours to once per day). The material is preferably free from impurities or bacteria that may render it unsafe for human use.

HA is one of the GAGs naturally present in the matrix of human lung. It plays a number of roles, including acting as a lubricant, and interacting with various cells and molecules in the lung environment. It is secreted by mesothelial cells in response to congestive heart failure, acute respiratory distress syndrome (ARDS), and other respiratory tract abnormalities. As used herein, the term HA means hyaluronic acid and any of its hyaluronate salts, including, for example, sodium hyaluronate (the sodium salt), potassium hyaluronate, magnesium hyaluronate, and calcium hyaluronate.

HA is a polymer consisting of simple, repeating disaccharide units. These repeating disaccharide units consist of glucuronic acid and N-acetyl glycosamine. It is made by connective tissue cells of all animals, and is present in large amounts in such tissues as the vitreous humor of the eye, the synovial fluids of joints, and the rooster comb of chickens. One method of isolating HA is to process tissue such as rooster combs. This invention can utilize HA isolated and purified from natural sources, as described in the prior art; HA isolated from natural sources can be obtained from commercial suppliers, such as Biomatrix, Anika Therapeutics, ICN, and Pharmacia.

Another method of producing HA is via fermentation of bacteria, such as streptococci. The bacteria are incubated in a sugar rich broth, and excrete HA into the broth. HA is then isolated from the broth and impurities are removed. The molecular weight of HA produced via fermentation may be altered by the sugars placed in the fermentation broth. This invention can utilize HA produced by bacterial fermentation as described in the prior art; HA produced via fermentation can be obtained from companies such as Bayer, Genzyme, and Lifecore Biomedical.

In its natural form, HA has a molecular weight in the range of 5×10⁴ up to 1×10⁷ Daltons. HA is soluble in water and can form highly viscous aqueous solutions. Its molecular weight may be reduced via a number of cutting processes such as exposure to acid, heat (e.g. autoclave, microwave, dry heat), or ultrasonic waves.

HA obtained from either animal tissue (e.g. rooster combs) or bacterial fermentation may contain contaminant proteins. Inhalation of protein contaminants may induce an allergic reaction in certain patients, causing bronchoconstriction, edema, and influx of inflammatory cells to the lung. Therefore, the HA of the invention have a protein content of less than 5%, more preferably less than 2%, and most preferably from 0% to undetectable levels. HA preparations may also contain endotoxin contaminants. To minimize the risk of an allergic reaction, the HA of the invention have an endotoxin concentration of less than 0.07 EU/mg, and preferably less than 0.01/EU/mg, and most preferably from 0% to undetectable levels.

The polysaccharides may serve as medium for bacterial growth. To insure that delivery of polysaccharides to the lung does not induce pneumonia, the material should be sterile.

Other physiologic parameters of the polysaccharides for use in the lung include pH between 4.0 to 8.9, and nontoxic concentrations of heavy metals, as judged by the criteria established for USP water for inhalation.

In one mode of the invention, a liquid formulation of polysaccharides is used. The liquid may be aerosolized for inhalation as a mist via an aerosolization device such as a nebulizer, atomizer, or inhaler.

In accordance with another mode, the formulation is a dry powder which individuals would mix at home or the hospital with saline or water before instillation to an aerosol device. The device would produce an aerosol for inhalation by the patient. A dry powder formulation could also be delivered in powder form by an aerosol device, such as air gun powered aerosol chamber. Companies which produce dry powder delivery devices include Dura Delivery Systems (the “Dryhaler”), Inhale Therapeutics, and Glaxo Wellcome (Diskhaler).

The respiratory system consists generally of three components: the tracheal/pharyngeal, the bronchial and the alveolar. It is known that particles of 10-50 microns migrate to the tracheal/pharyngeal component. Particles of about 5-10 microns migrate to the bronchial component, and particles of 0.5 to 5 microns migrate to the alveolar component. Particles less than 0.5 microns in size are not retained.

The mass median aerodynamic diameter (MMAD) is predictive of where in the lung a given particle will end up. The MMAD is usually expressed in microns. A related parameter is the geometric standard deviation (GSD). A GSD of 1 is equal to a normal distribution. A GSD of less than one indicates a narrow size dispersion and a GSD of more than 1 indicates a broad size dispersion.

Chemical modifications of polysaccharides may be used to produce new compounds which can bind to lung elastic fibers with an increased affinity. Elastin is a cationic protein. Consequently, introducing negatively charged groups, ions or substitutions can enhance the electostatic forces between the polysaccharide and the elastic fibers. For example, sulfate groups could be added to make the compound more negatively charged.

Various specific chemical modification schemes for HA are provided below. One skilled in the art could readily adapt these schemes to modify other polysaccharides.

Sulfate can be introduced to HA's hydroxyl groups, especially the 6-hydroxyl of the N-acetylglucosamine moiety, by the following reactions:

Reaction of tetrabutylammonium salt of HA with SO₃-pyridine as detailed in U.S. Pat. No. 6,027,741, entitled “Sulfated hyaluronic acid and esters thereof”; incorporated herein in its entirety by reference thereto.

Reaction of dry HA with chlorosulfonic acid in dry pyridine, as described by Wolfrom, M L, “Chondroitin sulfate modifications” J. Am. Chem. Soc. 82, 2588-2592.

Another means of adding sulfate groups to HA involves reaction with NH₂ after deacetylation of N-acetyl. The sulfation is completed in two steps, (a) deacetylation of N-acetylglucosamine moeity of HA by its reaction with anhydrous hydrazine at elevated temperature, followed by (b) treatment of the derived product with trimethylamine-sulfur trioxide. See e.g., U.S. Pat. No. 5,008,253, entitled “Sulfoamino derivatives of chondroitin sulfates of dermatan sulfate and of hyaluronic acid and their pharmacological properties”; the disclosure of which is incorporated herein in its entirety by reference thereto.

In addition to sulfate groups, carboxyl groups can be added to polysaccharides to increase their negative charge, thereby improving their binding to elastin in the lung matrix. The following reactions are provided to illustrate carboxylation schemes reactions for HA:

The 6-hydroxyl of the N-acetylglucosamine can be a target for further modification to introduce an additional carboxyl group, for example, reaction of dry HA with sodium chloroacetate.

The hydroxyl functional groups of HA are esterified by converting the carboxyl functional groups of HA into a tertiary ammonium or tertiary phosphonium salt in the presence of water and aprotic solvent and then treating the solution with succinic anhydride, as disclosed in U.S. Pat. No. 6,017,901, entitled “Heavy metal salts of succinic acid hemiesters with hyaluronic acid or hyaluronic acid esters, a process for their preparation and relative pharmaceutical compositions.

Similar to the previous example, dianhydrides such as ethylenediamine tetraacetic acid dianhydride (EDTAA) can be used. This reaction produces crosslinked HA. However, free pendant carboxyl groups from the anhydride may exist after the reaction of dianhydrides and HA, as described in U.S. Pat. No. 5,690,961, entitled “Acidic polysaccharides crosslinked with polycarboxylic acids and their uses”. Each of the above references are incorporated in their entirety by reference thereto.

Lipophilic side chains can also be attached to polysaccharides to increase the binding strength between the polysaccharide and elastin. Polar functional groups such as carboxyl and hydroxyl groups impart hydrophilicity. The introduction of lipophilic moieties to the polysaccharide can improve their affinity for elastin fibers, because elastin has a composition that is rich in amino acids with aliphatic side chains. The following reaction schemes are provided with respect to HA:

The introduction of an acetyl group to HA at its four hydroxyl site produces acetylhyaluronate. A method of manufacturing acetylhyaluronate comprises the steps of suspending hyaluronic acid powder in an acetic anhydride solvent and then adding concentrated sulfuric acid thereto to effect acetylation. The maximum degree of substitution is four, since there are four hydroxyl groups in each dissacharide unit of HA. Practically, only partial acetylation occurs. The degree of substitution determines the lipophilicity (thus hydrophobicity) of the modified HA. The more lipophilic, the higher the affinity of HA derivatives to the lipophilic moiety of elastin fibers. See e.g., U.S. Pat. No. 5,679,657, entitled “Low molecular weight acetylhyaluronate, skin-softening composition, method of manufacturing the same, and method of purifying the same”.

HA can react with alkylhalide, such as propyl iodide to form the ester function from the carboxyl group. The HA derivatives are less water-soluble and more lipophilic, proportional to the increase of degree of derivatization, as described in European Patent Application No. 86305233.8.

The reactions of free hyaluronic acid and diazomethane produce the methyl ester of HA, as described by Jeanloz et al., J. Biol. Chem. 186 (1950), 495-511.

Carbodiimides with aliphatic or aromatic side chains react with the carboxyl group of hyaluronic acid to form acylurea derivatives of HA with hydrophobic features, as described by Kuo et.al, Bioconjugate Chemistry, 1991,2, 232-241. Each of the above references is incorporated herein in their entirety by reference thereto.

In a preferred aspect of the present invention, a molecular weight of the polysaccharide or derivative is selected to produce a desired physiologic effect or molecular interaction, i.e., a desired therapeutic profile. As discussed above, the polysaccharides and their derivatives are polymers of repeating units and as a result, may be isolated, purified, synthesized, and/or commercially obtained in a wide range of molecular weights. The physiologic effects and molecular interactions of the polymers vary with molecular weight. Likewise, the physical delivery of the polymers to a selected target site within the lung also varies with polymer size (molecular weight). Different therapeutic profiles would be desirable for different clinical indications, and can be individually developed and optimized without undue experimentation by a physician skilled in the art, using the teachings disclosed herein.

For example, where protection of extracellular matrix against damage is desired, a high molecular weight preparation of polysaccharide would be desirable in order to provide effective binding to and coating of elastin fibers. Indeed, a high molecular weight polysaccharide derivative, modified to enhance its affinity for elastin, would be preferred. High molecular weight preparations are also preferred for depot of drugs, where the large polymer may be a better excipient, a better carrier and better for addressing large airway diseases. Alternatively, lower molecular weight preparations may be better for loosening sputum, penetrating to the deep lung tissues, and traversing alveolar-epithelial barrier. In selecting the molecular weight, the physician will have to balance the desired therapeutic profile against physical restraints on delivery into the deep lungs.

With respect to duration in the lungs, a polymer preparation in accordance with the present invention may have a molecular weight that resides in the lung for between 0.5 hour and one week, preferably between 1 hour and one day, and more preferably between 4 and 16 hours. Most preferably, a GAG will remain associated with the lung matrix for at least 6 hours. This would allow for dosing four or less time a day.

It has been observed that molecular weights of HA preparations for between 25,000 Daltons and 2,000,000 Daltons can be used to provide lung duration times, water retention, elastic recoil, and matrix coverage, consistent with the above. The relationship between polysaccharide concentration, molecular weight and viscosity is discussed in greater detail below. When a preparation of HA having a molecular weight of greater than 2,000,000 Daltons was used, it produced a solution that was excessively viscous. Thus, although the highest molecular weight preparations yield the greatest duration times, water retention, elastic recoil and matrix coverage, these properties must be balanced against excessive viscosity, particularly at lower deployment temperatures (e.g., jet nebulizers that cool the solutions significantly during expansion). In general, it has been observed for HA, that it was preferred to use a preparation having a molecular weight of less than about 1.5×10⁶ Daltons, more preferably less than 500 kD, more preferably still, less than about 220 kD, and most preferably less than about 150 kD.

Besides the molecular weight, the concentration of the glycosaminoglycan solution also influences duration times, water retention, elastic recoil, and matrix coverage, and formulation viscosity. Viscosity increases with increasing concentration. Viscosity increases with decreasing temperature. Concentrations of HA are preferably between about 0.05 mg/L and 5 mg/L at ambient temperature (20° to 25° C.). The preferred concentration is less than 5 mg/L, more preferably less than 2 mg/L, and more preferably less than 1 mg/L. The preferred concentration is above 0.05 mg/L, more preferably over 0.5 mg/L. The concentration of a selected molecular weight preparation may be adjusted to yield a selected viscosity, depending on the temperature.

The viscosity or thickness of the material is related to the combination of concentration and molecular weight. Viscosity increases with increasing molecular weight if concentration remains constant. Likewise, viscosity increases with increasing concentration if molecular weight remains constant. Viscosity can be measured by a viscometer (one such device is manufactured by the company Brookfield), and is expressed in units of centipoises (abbreviation: cps).

The material must be transferred from the delivery device (e.g. via an aerosolization device) into the respiratory tract, down to the distal bronchi and alveoli, from where it can diffuse into the extracellular lung matrix. The delivery formulation should have physical characteristics which avoid clogging of the aerosol device and clumping of aerosolized particles. It should be noted that a viscous material, delivered slowly, may not cause clogging or plugging, whereas a less viscous material may, if delivered quickly.

Formulations of specific molecular weight, concentration and viscosity are preferably produced by adding a volume of sterile delivery solvent (e.g., water or saline) to an amount of sterile, medical grade polysaccharide powder. More preferably, unit dose vials containing a pre-weighed dose of polysaccharide may be dissolved just prior to use by injection of sterile solvent into the sealed vial. The powdered polysaccharide is then mixed in the solvent until dissolved. Alternatively, polysaccharide of a certain concentration can be prepared by diluting liquid polysaccharide with sterile solvent.

Formulation temperatures of between about 0 to about 100° C., preferably between about 4° and 60° C. and more preferably between about 15° and 37° C. may be used in accordance with the present invention; however, the viscosity of a given molecular weight and concentration of a polysaccharide varies with temperature. Thus, the user can determine empirically the viscosity with a viscometer, and adjust the concentration accordingly to yield a viscosity adapted for delivery by the desired delivery mechanism (e.g., nebulizer, aerosolizer, inhaler etc.) to the selected target site in the lungs. For delivery to the lungs at ambient temperature, the viscosity is preferably below about 1,000 cps, more preferably below about 100 cps, and most preferably below about 50 cps.

Another factor which should be considered in formulating a polysaccharide solution for delivery to a selected target site in the respiratory tract is the droplet or particle size generated. This factor should be considered for aerosol as well as powder delivery pathways. Particle size is preferably below about 10 microns in diameter. More preferably, the particle size is between 2 and 5 microns. The relationship between particle size in microns and fluorescence-labeled polysaccharide molecular weight and concentration can be measured as the Mass Median Aerodynamic Diameter using a Cascade Impactor (see data in Examples below). The numbers on the x-axis represent sieve sizes in microns and the numbers on the y-axis represent fluorescence (i.e., amount of polysaccharide) which impacts on the particular sieve (i.e., median particle size is too large to fit through the pores). A humidified variation of the Cascade Impactor can also be used to more closely reflect pulmonary delivery, because the polymers of the present invention may be hydroscopic and therefore absorb water and swell in size.

Raabe et al., reported a survey of particle size access to various airways in small laboratory animals using inhaled monodisperse aerosol particles. Raabe et al., Ann. Occup. Hyg. 1988, 32:53-63; incorporated herein by reference thereto. Similar analysis may be performed to inform the clinician as to the desirable particle size for delivery to a target site within the lung.

Particle size in accordance with a preferred mode of the present invention may be between about 2 microns and about 5 microns, thereby being adapted for delivery into the lung alveoli. Larger size particles are not as efficiently delivered through the distal bronchioles, whereas much smaller sizes tend to be exhaled before contacting the alveolar lining. Thus, whereas the therapeutic profile (e.g., duration, water retention, elastic recoil and matrix coverage) tend to increase with increasing molecular weight, the relative deliverability (i.e., frequency of particles within the 2-5 micron range) tends to decrease with increasing molecular weight.

In order to produce an aerosol which can be inhaled by human beings for distribution throughout the lung, the glycosaminoglycan must be aerosolized into appropriate droplet sizes as detailed above, preferably between about 2-5 microns in diameter. Some droplets larger than 5 microns in diameter may deposit in the nebulizer tubing or mask, mouth, pharynx, or laryngeal region. Droplets less than 2 microns in diameter tend not to be deposited in the respiratory tract, but are exhaled and lost. Droplet sizes of 2-5 microns can be achieved by selection of appropriate aerosol devices, solution concentration, compound molecular weight, and additives, in accordance with the teachings herein.

Additives such as surfactants, soaps, Vitamin E, and alcohol may be added to avoid clumping of droplets after they are produced, and to facilitate generation of small particles from an aerosol device. One embodiment of the invention includes glycosaminoglycans in combination with one or more of these additives.

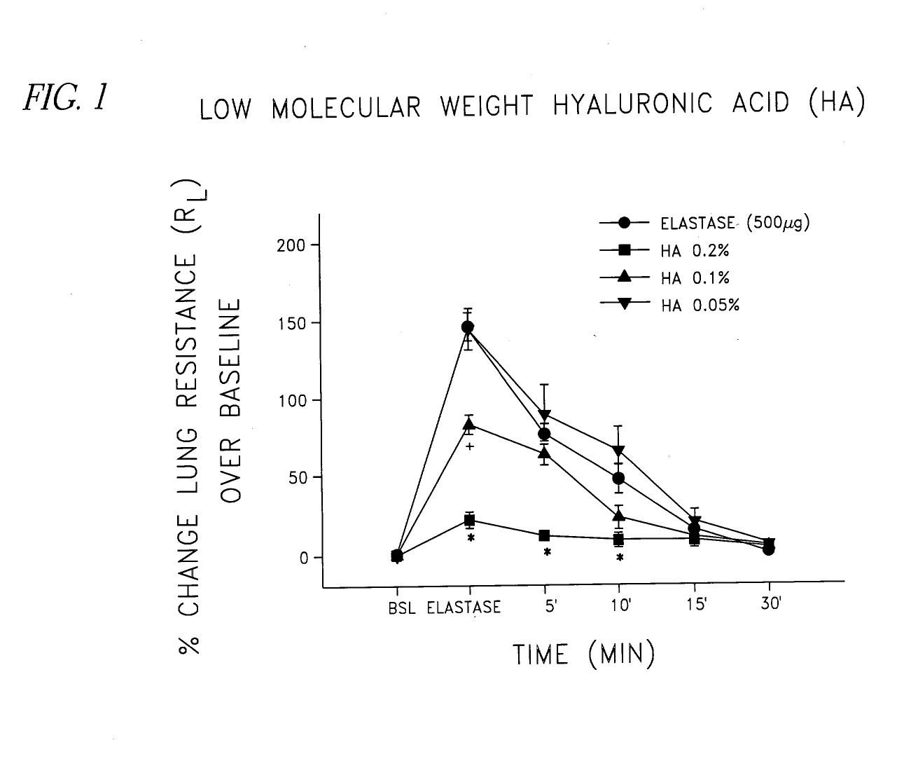

A method of selecting breathable formulations for delivery to the lung by aerosol is to screen multiple formulations for those formulations which will produce droplets of less than 10 microns in diameter, more preferable less than 6 microns, most preferably 2-5 microns. Formulations which produce droplets larger than 10 microns are not suitable for delivery into the lung. Particle size distribution of the aerosolized mist for each formulation is measured with a device such as a Malvern Laser or a Cascade Impactor (as used to generate the data shown in FIGS. 1A-L). This invention includes all molecular weight and concentration combinations of polysaccharides that can be aerosolized into droplet sizes of under 10 microns, and more preferably between about 2-5 microns.

One embodiment of the invention involves use of an aerosol-generating device to produce an inhalable mist. One class of device to generate polysaccharide aerosols is a spray atomizer. Another class of device to generate polysaccharide aerosols is a nebulizer. Nebulizers are designed to produce droplets under 10 microns.

Many commonly used nebulizers may be used to aerosolize polysaccharides for delivery to the lung: 1) compressed air nebulizers (examples of these include the AeroEclipse, Pari L. C., the Parijet and the Whisper Jet) and 2) ultrasonic nebulizers. Compressed air nebulizers generate droplets by shattering a liquid stream with fast moving air. One mode of the invention involves use of a compressed air nebulizer to aerosolize polysaccharide solutions into droplets under 10 microns in size. Ultrasonic nebulizers use a piezoelectric transducer to transform electrical current into mechanical oscillations, which produces aerosol droplets from a liquid solution. Droplets produced by ultrasonic nebulizers are carried off by a flow of air. Another mode of the invention involves the use of an ultrasonic nebulizer to aerosolize polysaccharide solutions into droplets less than 10 microns in size.

Another mode of this invention is use of a hand-held inhaler to generate polysaccharide aerosols. This portable device will permit an individual to administer a single dose of mist, rather than a continuous “cloud” of mist into the patient's mouth. Individuals with bronchoconstrictive diseases such as asthma, allergies, or COPD often carry these hand-held inhalers (e.g., MDI and DPI) in their pocket or purse for use to alleviate a sudden attack of shortness of breath. These devices contain bronchodilator medication such as albuterol or atrovent. They would also be a convenient way to deliver glycosaminoglycan to patients.

For treatment via nebulizer, patients would inhale the aerosolized polysaccharide solution via continuous nebulization, similar to the way patients with acute attacks of asthma or emphysema are treated with aerosolized bronchodilators. The aerosol may be delivered through tubing or a mask to the patient's mouth for inhalation into the lungs. Treatment time may last 30 minutes or less. The mouth is preferably used for inhalation (rather than the nose) to avoid “wasted” nasal deposition. To optimize the delivery rate of polysaccharide via nebulizer, the volumetric flow rate (L/min) of the nebulizer preferably does not exceed two times the patient's minute ventilation, although this can be varied depending on the polysaccharide formulation and the clinical status of the patient. This is because the average inspiratory rate is about twice the minute ventilation when exhalation and inhalation each represent about half of the breathing cycle. In one mode of the invention, a nebulizer with a volumetric flow rate of under 15 L/min is employed.

The particle size distribution generated from nebulizers is a function of a number of variables related to the nebulizer as well as the formulation (as discussed above). Nebulizer related factors for compressed air nebulizers include air pressure, air flow, and air jet diameter. Nebulizer related factors for ultrasonic nebulizers include ultrasound frequency, and rate/volume of air flow. In one mode of the invention, a compressed air nebulizer with specific air pressure, air flow, and hole diameter settings is used to generate droplets of a specific polysaccharide formulation under 10 microns. In another mode, an ultrasonic nebulizer with specific frequency and hole diameter settings is employed to generate droplets of a specific polysaccharide formulation under 10 microns.

Other considerations that determine selection of an ideal nebulizer and formulation include solution use rate (ml/min), aerosol mass output (mg/L), and nebulizer “hold up” (retained) volume (ml). The interaction among these factors will be appreciated by those of skill in the art.

Aerosolized polysaccharide could be delivered from nebulizer to a patient's respiratory tract via face mask, nonrebreather, nasal cannula, nasal covering, “blow by” mask, endotracheal tube, and Ambu bag. All of these connections between the patient and nebulizer are considered to fall within the scope of the present invention.

In addition to delivery via unassisted inhalation, another embodiment of the invention involves delivery of aerosolized polysaccharides under positive pressure ventilation. A commonly used ventilatory assist device is CPAP: Continuous Positive Airway Pressure. In this application, a breathing mask is sealed around the mouth of a patient. The patient is then administered oxygen through the mask at a certain pressure to facilitate inspiration. Delivery of polysaccharides through a CPAP mask might enhance delivery of material to the deep airways. To facilitate delivery to the alveoli and transfer across the alveolar epithelial barrier, the polysaccharide could be delivered while the patient is being ventilated with positive end expiratory pressure (PEEP).

Another mode of the invention is to deliver aerosolized polysaccharides with a device that delivers material when the patient generates a certain level of negative inspiratory pressure.

Another mode of the invention is to deliver polysaccharides in conjunction with ventilation through an endotracheal tube. One benefit of this embodiment is to protect against oxygen toxicity in patients ventilated with high concentrations of oxygen. In addition the viscoelastic properties of polysaccharides should protect the lungs from ventilator associated barotrauma that results in the complication of pneumothorax.

Given that this invention is a nontoxic therapy, which exerts its beneficial effects in respiratory disease by its physical presence in the lung, the formulation of this invention should allow for the polysaccharide to remain in the lung continuously. The half-life of HA injected in the pleural (potential space between the lung and the chest wall) of rabbits has been shown to range between 8 and 15 hours. The half-life is longer if more HA is injected. Commonly inhaled medications for emphysema are used from one to three times a day. More frequent dosing requirements present a compliance issue with patients. One aspect of this invention involves a formulation of polysaccharide that resides in the lung for 6 hours to be given 4 times per day, or preferably for 8 hours, to be given three times per day. A more preferable embodiment is a formulation that remains in the lung for 12 hours, which will be administered twice a day. A more preferable embodiment is a formulation that remains in the lung for 24 hours, which will be administered once a day.

The effect of different formulations on duration is studied in mammals by tagging the polysaccharide with a radiolabel such as tritium, C¹⁴, Thallium, or Technecium. Alternatively, a direct assay for the particular polymer could be employed. One radiometric assay for HA uses ¹²⁵I-labeled HABP (HA binding protein); this assay is commercially available from Pharmacia (“Pharmacia HA Test”). Material is delivered to the lungs and monitored over time by use of a scintillation counter (e.g. gamma camera). Alternatively, a group of animals (e.g. rats) is given radiolabeled-glycosaminoglycan in the lungs and then serially sacrificed over time. Excised lung tissue is examined for radioactivity, and duration time or half-life is determined.

Just as the invention encompasses protecting the lungs with aerosol polysaccharide, the invention also encompasses application of polysaccharide by aerosol delivery to other tissues, including for example, exposed tissues during surgery, sinus passageways, burns, and mucous membranes.

### STUDY 1

A total of 9 sheep (mean weight: 30.5 Kg) were used for this study. All animals had a history of airway sensitivity to inhalation of Ascaris Suum antigen. The study was conducted at Mount Sinai Medical Center under the approval of the Mount Sinai Medical Center Animal Research Committee.

Airway Mechanics

To study the elastase-induced changes in airway mechanics, the animals were restrained in a cart, in an upright position with their heads immobilized. A balloon catheter was advanced through one nostril into the lower esophagus after topical anesthesia with 2% lidocaine solution. The animals were intubated with a cuffed endotracheal tube through the other nostril, using a flexible fiberoptic bronchoscope. Pleural pressure was measured via an esophageal catheter (filled with 1 ml of air) positioned 5 to 10 cm from the gastroesophageal junction. In this position the end expiratory pleural pressure ranged between −2 and −5 cm H₂O. Lateral pressure in the trachea was measured with a sidehole catheter (inner dimension, 2.5 mm) advanced through and positioned distal to the tip of the endotracheal tube. Transpulmonary pressure, the difference between tracheal and pleural pressure, was measured with a differential pressure transducer catheter system. For the measurement of pulmonary resistance (R_{L}), the proximal end of the endotracheal tube was connected to a pneumotachograph (Fleisch; Dyna Sciences, Blue Bell, Pa.). The signals of flow and transpulmonary pressure were recorded on an oscilloscope recorder, which was linked to a computer, for on-line calculation of R_{L}. Respiratory volume was obtained by digital integration of the flow signal and was used, together with transpulmonary pressure and flow, at isovolumetric points to derive R_{L} (as described by Von Neergaad K et al. 1927 Z. Klin. Med. 105:51-82), as previously described (Forteza R et al. 1996 Am. J. Resp. Crit. Care Med. 154:36-42; incorporated herein in its entirety by reference). Analysis of 5-10 breaths was used for the determination of R_{L}.

Aerosols

Aerosols were generated using a disposable medical nebulizer (Raindrop; Puritan Bennett, Lenexa, Kans.). The output from the nebulizer generated an aerosol with mass median aerodynamic diameter of 3.2 μm (geometric SD 1.9) as determined by an Andersen cascade impactor. The output of the nebulizer was directed into a plastic T-piece, which was interconnected to the inspiratory port of a Harvard piston ventilator (Harvard Apparatus, Natick, Mass.) with the animal's tracheal tube. To control aerosol delivery, a dosimeter system consisting of a solenoid valve and a source of compressed air (20 psi) was used. The solenoid valve was activated for 1 second at the beginning of the inspiratory cycle of the ventilator. Aerosols were delivered at a tidal volume of 500 ml and a rate of 20 breaths/min.

Agent

Porcine pancreatic elastase (PPE) was purchased from Sigma Aldrich Co. (St. Louis, Mo.), dissolved in phosphate buffered saline (PBS; pH 7.4) to a stock concentration of 5 mg/ml. Aliquots of 500 μg were kept at −20° C., dissolved in 3 ml PBS (pH 7.4) the day of the experiment and delivered as an aerosol (20 breaths/min×20 min). LMW-HA from pig trachea (avg. molecular weight about 70K Daltons) was purchased from Fluka Chemical Corp. (Milwaukee, Wis.), dissolved in distilled water to a 1% stock solution and then diluted in PBS (3 ml; pH 7.4) to a concentration of 0.2, 0.1, and 0.05% the day of the experiment. HMW-HA from human umbilical cord (avg. molecular weight about 200K Daltons) was purchased from ICN Biomedicals, Inc. (Aurora, Ohio), dissolved in distilled water to a 1% stock solution and then diluted in PBS (3 ml; pH 7.4) to a concentration of 0.05, 0.01, and 0.005% the day of the experiment. All solutions were delivered as an aerosol (20 breaths/min×20 min).

Bronchoalveolar Lavage for Tissue Kallikrein Analysis

The distal tip of a specially designed 80 cm fiberoptic bronchoscope was wedged into a randomly selected subsegmental bronchus. Lung lavage was performed by slow infusion and gentle aspiration of 60 ml of PBS (pH 7.4 at 37° C.) in two different airway segments (30 ml each), using a 30 ml syringe attached to the working channel of the instrument. The effluent was filtered through a double layer of gauze and transferred into a tube. All tubes were placed immediately on ice and then centrifuged at 250×g at 4° C. for 15 minutes. The supernatant was recentrifuged at 3000×g at 4° C. for 15 minutes, saved and frozen at −80° C. for subsequent analysis.

Analysis of Bronchoalveolar Lavage Fluid (BALF)

Before mediator analysis, BALF supernatant was thawed and recentrifuged at 12,500×g at 4° C., for 15 minutes. Unconcentrated BALF supernatant was analyzed for TK activity by cleavage of DL Val-Leu-Arg pNA as described previously (Forteza R et al. 1996 Am. J. Resp. Crit. Care Med. 154:36-42; incorporated in its entirety by reference) and was expressed as arbitrary units (1 Unit=change in optical density at 405 nm in 24 hours).

Effect of Inhaled Hyaluronic Acid (HA) on Elastase-Induced Bronchoconstriction.

Six animals were challenged with inhaled elastase (PPE 500 μg, in 3 ml PBS; pH 7.4). In the control protocol, elastase was given 30 min after placebo (PBS, 3 ml; pH 7.4). R_{L} was measured before, immediately after and at 5, 10, 15, and 30 min after challenge. In the treatment protocol, elastase was given 30 min before either inhaled LMW-HA (3 ml in PBS; pH 7.4) at concentrations of 0.2, 0.1, and 0.05%, or HMW-HA (3 ml in PBS; pH 7.4) at concentrations of 0.05, 0.01, and 0.0005%). RL was measured, before, immediately after and at 5, 10, 15, and 30 min after challenge. Each experiment was separated by at least 72 hours.

Effect of HA on Elastase-Induced TK Activity in BALF

BALF TK activity was measured at baseline and 30 minutes after challenging the animals with inhaled elastase (PPE 500 μg). The same procedure was repeated after pretreatment with HMW-HA at concentrations of 0.05, and 0.005%.

Statistics.

All data were analyzed using a multivariate analysis of variance (ANOVA) for repeated measures followed by post-hoc t-test with Bonferroni correction to identify significant pairs. Individual comparisons were made using paired and unpaired t-test when appropriate (Sigmastat 2.0 for Windows, SPSS Inc., Chicago, Ill.). Values in the text and figures are presented as mean±SE; p<0.05 was considered significant.

Effect of HA on Elastase-Induced Bronchoconstriction.

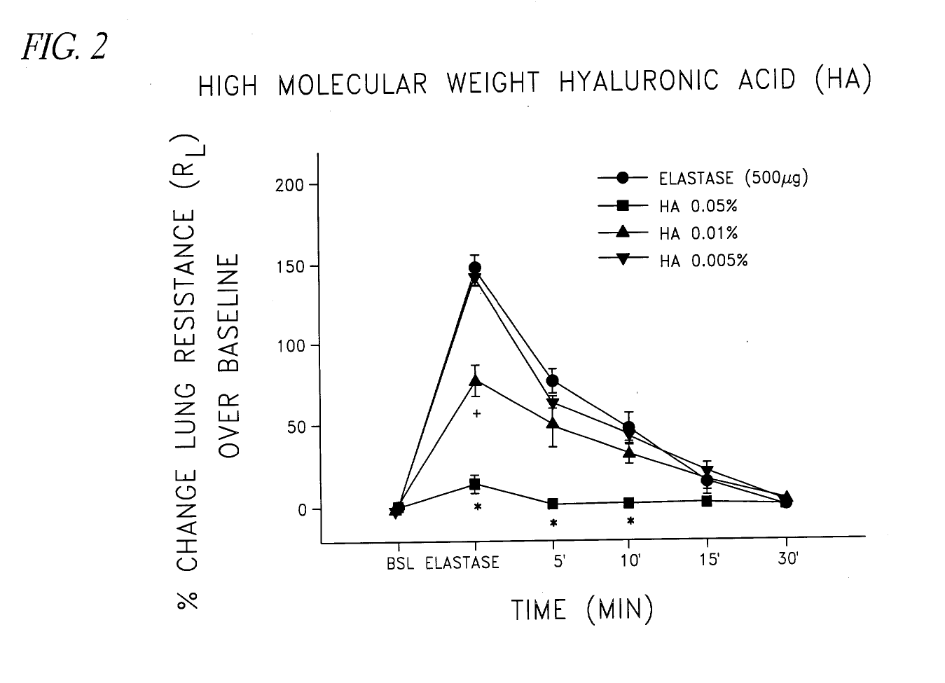

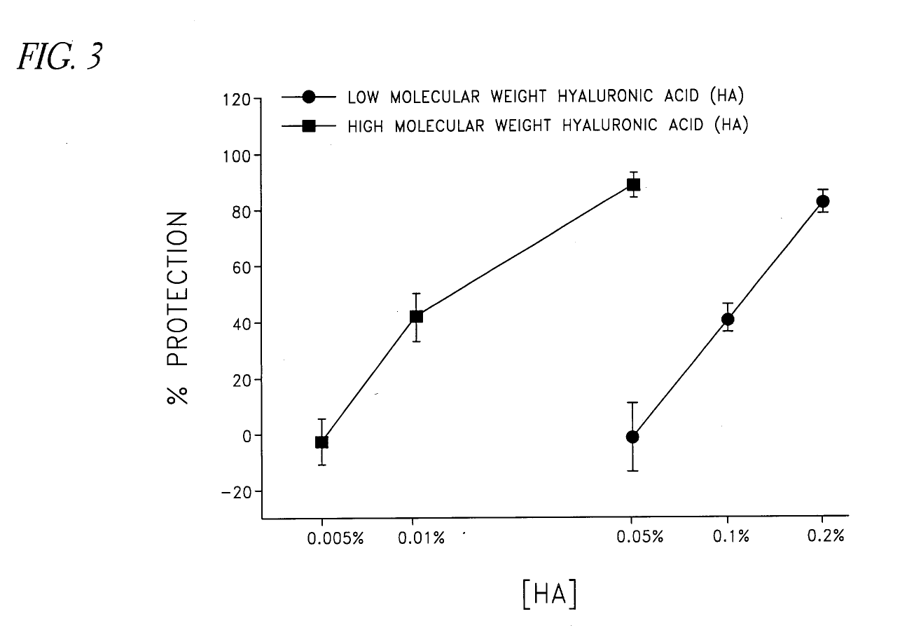

Inhaled elastase (500 μg) caused a short-lived bronchoconstriction reaching its peak immediately after challenge to resolve within 30 minutes. Pretreatment with aerosolized LMW-HA (0.2%) completely blocked this response (p<0.001; n=6) whereas inhalation of lower doses (0.1% and 0.05%) resulted in a differential protection against elastase-induced bronchoconstriction indicating a dose-related effect (FIG. 1). When the animals were pretreated with HMW-HA, complete protection against elastase-induced bronchoconstriction was achieved at a much lower dose (0.05%; p<0.001, n=6). Aerosolization of lower doses of HMW-HA (0.01, 0.005%), again, showed a dose-dependent effect (FIG. 2). FIG. 3 illustrates the dose-dependent and molecular weight-dependent effects and shows that the higher the molecular weight of HA, the higher the degree of protection achieved against elastase-induced bronchoconstriction.

Effect of HA on Elastase-Induced TK Activity in BALF.

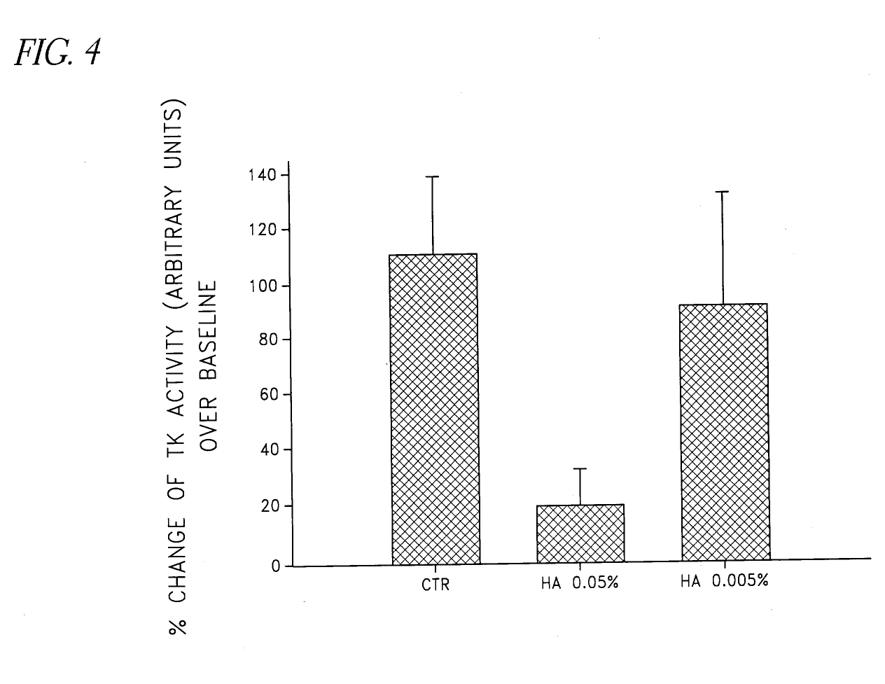

Consistent with the physiologic data, elastase (500 μg) induced a significant increase in BALF TK activity (p<0.05; n=8) 30 min after challenge. This increase was inhibited by pretreatment with inhaled HMW-HA 0.05% (p<0.05; n=7), whereas inhaled HMW-HA 0.005% was ineffective (FIG. 4).

The results of this study show that inhaled HA prevents the elastase-induced bronchoconstriction in a dose-dependent and molecular weight-dependent fashion. This protection is associated with inhibition of TK activity in BALF of allergic sheep. In the allergic sheep model, inhaled elastase increased lung TK activity and caused bronchoconstriction via the formation of bradykinin (Scuri M et al. 2000 J. Appl. Physiol. 89(4):1397-1402; incorporated herein in its entirety by reference thereto). Further, bronchial TK bound to HA thereby reducing its activity in vitro (Forteza R et al. 1999 Am. J. Resp. Cell Mol. Biol. 21:666-674; incorporated herein in its entirety by reference thereto). The molecular weight-related effect was unexpected. An explanation for this probably lies in the structure of HA itself and may explain this result. HA is a long polymer and, although the TK binding site has not yet been characterized, it is conceivable that a heavier and thus longer HA molecule, carries more binding sites for TK. Thus, it is possible that HMW-HA can bind more TK molecules at any given concentration and so provide better protection against the elastase-induced airway responses.

Some reports claim that LMW-HA causes the induction of inflammatory factors via a CD44-mediated mechanism (Noble P W et al. 1998 In: The chemistry, biology and medical applications of hyaluronan and its derivatives. London, Portland Press, pgs. 219-225). In this study, however, no inflammatory response was observed in the animals that received LMW-HA. This observation is consistent with data from Lackie et al. (Lackie P et al. 1997 Am. J. Resp. Cell Mol. Biol. 16(1):14-22), who showed that CD44-mediated actions in the airways are associated with repair rather than with inflammatory processes.

In order to provide biochemical support for the functional in vivo data, the protective effect of HA was measured against the elastase-induced increase in BALF TK activity. For these studies HMW-HA was used at two different concentrations: one that was effective in blocking the elastase-induced bronchoconstriction (0.05%) and one that was ineffective (0.005%). The mediator data supported the functional ones with 0.05% HMW-HA suppressing the increase in TK while 0.005% HMW-HA failed to do so. Collectively, these data support the concept that the effects of HA are mediated by its binding and inhibition of BALF TK activity.

The biologic reason for TK to be bound to HA is not known, but association of glycosaminoglycans (GAGs) with proteases and protease inhibitors can regulate their functions by different mechanisms including but not limited to: (1) enzyme immobilization, leading to the restriction of its range of action; (2) stearically blocking its activity; (3) providing a reservoir for delayed release; or (4) protecting it from proteolytic degradation (see e.g., Ying Q L et al. 1997 Am. J. Physiol. 272(3 Pt 1):L533-541). These GAGs-proteinase interactions could be similar for TK-HA interactions. Forteza et al. have previously shown that HA binding to TK blocks its enzymatic activity (Forteza R et al. 1999 Am. J. Resp. Cell Mol. Biol. 21:666-674). HA is elevated in BALF of asthmatic patients (Vignola A M et al. 1998 Am. J. Resp. Crit. Care Med. 157(2):403-409) indicating that its turnover is altered in these subjects. In vitro, human neutrophil elastase causes the release of TK from primary cultures of ovine tracheal gland cells as already shown in studies conducted in this laboratory (Forteza R et al. 1997 Am. J. Resp. Crit. Care Med. 155(4):A357). Moreover, inhaled elastase caused bronchoconstriction in allergic sheep via a bradykinin-mediated mechanism (Scuri M et al. 2000 J. Appl. Physiol. 89(4):1397-1402). Antigen challenge also increased free elastase activity in BALF of allergic sheep (O'Riordan T G et al. 1997 Am. J. Resp. Crit. Care Med. 155:1522-1528) which, in turn, stimulated TK release. Other stimuli such as cell products (Trahir J F et al. 1989 Histochem. Cytochem. 37:309-314; Sommerhoff S P et al. 1990 J. Clin. Invest. 85:682-689), sensory nerve stimulation (Gashi A A et al. 1986 Am. J. Physiol. 251:C223-C229) and autonomic stimulation (Culp D J et al. 1996 Am. J. Physiol. C1963-C1972) are well characterized secretagogues. All these mechanisms could be expected to increase TK release and, therefore, kinin generation when substrate is available, thus providing a positive stimulus for kinin-induced airway inflammation. TK is also thought to be a mediator in rhinitis and asthma.

Bronchial TK was resistant in vitro to inhibition by most of the serine protease inhibitors present in BALF, suggesting that there was no effective inhibition for TK in the airways. However, as disclosed herein, HA does plays an important role in the regulation of bronchial TK activity by binding to it, thus preventing its biologic actions. This disclosure adds new evidence that, in vivo, exogenous HA can restore the physiologic HA-TK interaction, thus preventing the elastase-induced bronchoconstriction and increase in BALF kinins. It is likely that this anti-elastase effect of HA is based on enzymatic inhibition of TK. This represents the first observation of a functional protection of HA in the airways. Cantor et al. (Cantor J O et al. 1999 Connect. Tiss. Resp. Tech. 40(2)97-104) showed that HA prevents the elastase-induced emphysema in hamsters. This effect, however, depends on a mechanic property of HA, which forms a protective coating on the elastin in the lung, thus limiting its degradation by elastase released from neutrophil and/or macrophages. The results of these studies suggest that the protective effect of HA against elastase-induced lung injury is not related to an enzymatic interaction with TK, and thereby does not interfere with its regulation.

In conclusion, the results of these experiments support the functional protection of HA in the airways, which was unexpected based on the earlier work referenced herein. Furthermore, these results add new evidence that HA may play a role in regulating T′K activity in vivo, shedding a new light on the mechanism of action of this polysaccharide.

In summary, neutrophil elastase can cause release of TK from tracheal gland cells in allergic mammals. The increase in lung TK activity mediates the bronchoconstrictor response to inhaled elastase via the formation of bradykinin. HA bound airway TK, thereby reducing its activity in vitro. To test the hypothesis that HA would inhibit and/or prevent bronchoconstriction by binding TK, pulmonary resistance (R_{L}) was measured in allergic sheep before and after inhalation of elastase alone (500 μg) and after pretreatment with either inhaled low molecular weight HA (LMW-HA; 3 ml) or high molecular weight HA (HMW-HA; 3 ml) at different concentrations. Each treatment was separated by at least 72 h. Inhaled elastase increased R_{L} 147±8% (mean±SE) over baseline immediately after challenge. HA blocked the elastase-induced bronchoconstriction in a dose and molecular weight dependent fashion with 0.2% LMW-HA and 0.05% HMW-HA both providing a complete protection. HA alone had no effect on R_{L}. Consistent with the physiologic data, TK activity in the bronchoalveolar lavage fluid (BALF) increased 111±28% over baseline after challenge in inhaled elastase (500 μg). This response was inhibited by HMW-HA 0.05% but not by 0.005% HMW-HA, which was also ineffective in blocking the elastase-induced bronchoconstriction. Thus, HA blocks the elastase-induced bronchoconstriction in a dose-dependent and molecular weight-dependent fashion. These are the first data to show functional protection by HA in the airways.

### STUDY 2

Both airway lactoperoxidase (“LPO”) and tissue TK are key enzymes in airway mucosal defense. Airway LPO was purified as described (Salathe M et al. 1997 Am. J. Resp. Cell Mol. Biol. 17:97-105) and shown to stimulate bacterial clearance of the airways (Gerson C et al. 2000 Am. J. Resp. Cell Mol. Biol. 22:665-671). Bronchial TK mediates allergic bronchoconstriction and thereby limits the inhalation of noxious substances. Both enzymes are secreted from airway submucosal gland cells. It has been commonly believed that proteins are rapidly cleared by the mucociliary apparatus after secretion. Therefore, secretion has been postulated to be the main determinant of enzyme availability (and activity) on mucosal surfaces. The observations presented here, however, suggest that enzymes can be retained and regulated at the ciliary border of airway epithelial cells by binding to HA. This finding may apply to other mucosal surfaces and changes the way we have to think about secretion and enzyme availability.

To identify the localization of both airway LPO and bronchial TK, primary cultures of ovine airway epithelial cells were used containing submucosal gland cells and ovine tracheal sections, all fixed with acid formalin as described in order to preserve carbohydrates usually lost during tissue processing (Lin W et al. 1997 J. Histochem. Cytochem. 45:1157-63) Briefly, polyclonal rabbit anti-human urinary kallikrein serum (Calbiochem) has previously been demonstrated to recognize specifically bronchial TK. Antiserum to purified sheep airway LPO was made in rabbits (Covance, Hazelton, Pa.). Specificity was determined by Western blotting with purified sheep and bovine LPO as well as canine and human MPO. Rabbit anti-chicken IgG, used as a control in the CBF experiments, was from Cappel (Organon Teknika Corporation). Sheep trachea and cell cultures were fixed with acid formalin and processed according to standard procedures for immunohistochemistry and immunocytochemistry. Primary antibodies were used at the following dilutions: anti-TK (1:500); and anti-LPO (1:500). Pre-immune serum was diluted 1:500. Using affinity purified alkaline phosphatase or horse-radish peroxidase labeled goat anti-rabbit IgG (5 μgml in 50 mM Tris buffered saline; Kirkegaard & Perry) as secondary antibodies, color was developed with nitro blue tetrazolium (NBT) and 5-bromo-1-chloro-3-indolyl-phosphate (BCIP) and diaminobenzidine (DAB), respectively.

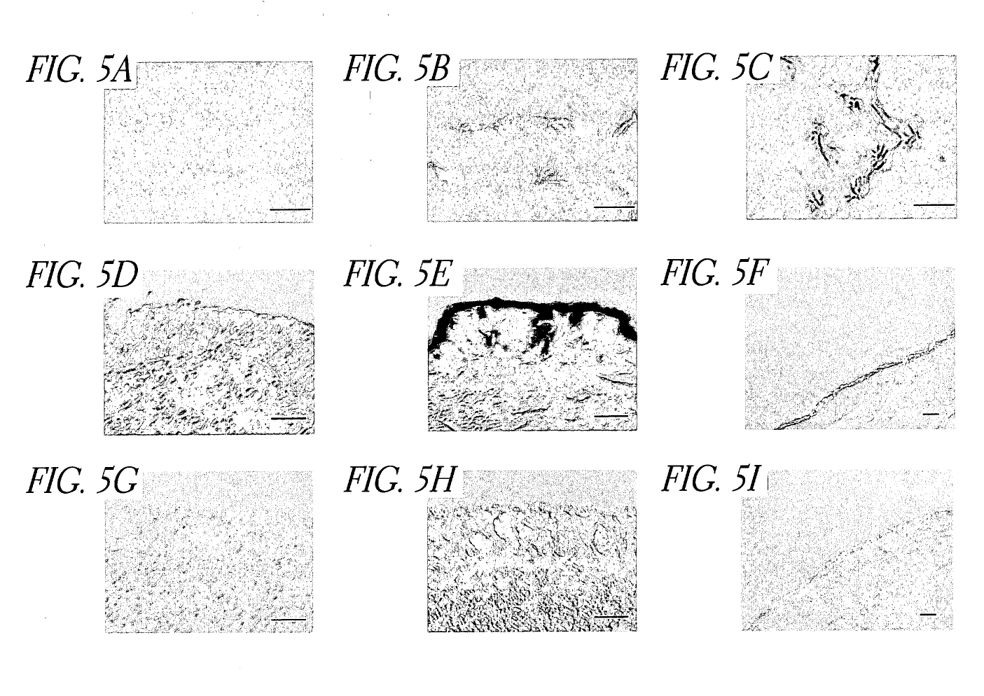

Surprisingly, immunocytochemistry of cultures showed specific staining for both enzymes on cilia (FIG. 5). Immunohistochemistry of tracheal sections revealed specific staining not only in submucosal gland cells for both enzymes and in goblet cells for LPO, but also along the ciliated border of the airway epithelium (FIG. 5). Pre-immune serum did not reveal any nonspecific staining in the ciliary border of tissue sections or cell cultures. In addition, direct visualization of LPO's activity in tissue sections using diaminobenzidine (Salathe M et al. 1997 Am. J. Resp. Cell Mol. Biol. 17:97-105) confirmed the results obtained by immunostaining, again ruling out non-specific adherence of antibodies to the ciliary border.

To determine whether these enzymes are immobilized at the apex of epithelial cells by binding to HA, immunohistochemistry of tracheal sections for HA was analyzed using a biotinylated HA-binding protein (Bray B A et al. 1994 Exp. Lung Res. 20:317-30). HA was visualized using a biotinylated HA-binding protein (Seikagaku). Hyaluronidase digestion was accomplished with hyaluronidase (50 U/ml at pH 5.5; Seikagaku) in a cocktail of protease inhibitors (pepstatin 10 μg/ml, aprotinin and leupeptin 10 ng/ml) in 50 mM Tris buffered saline, pH 5.5, at 37° C. overnight. The results shown in FIG. 5 indicate that the ciliated border of the epithelium was labeled. Digestion with hyaluronidase eliminated the apical staining for HA as well as LPO and TK (FIG. 5). This elimination was specific for HA because hyaluronidase did not remove glycoconjugates from the apical border of the epithelium (as evidenced by Alcian-blue-PAS staining).

After having previously shown that TK binds to HA using a non-denaturing gel system and affinity chromatography (Forteza R. et al. 1999 Am. J. Resp. Cell Mol. Biol. 21:666-74), the putative HA-binding motif B(X₇)B (Yang B. et al. 1994 Embo J. 13:286-96) was identified in the amino-acid sequence of TK, providing a basis for specific interactions between HA and TK. Airway LPO was also binding to HA, as determined by non-denaturing agarose gel electrophoresis. However, analysis of the airway LPO amino-acid sequence did not reveal the presence of known HA-binding motifs. Instead, LPO probably binds to HA because of its alkaline pI by ionic interaction. In fact, HA may act as a cation exchanger and may be able to bind several other cations to the epithelial surface. Among those could be a variety of cationic antimicrobial substances, for example those studied by Cole et al. (Cole A M et al. 1999 Infect. Immun. 67:3267-75).

HA binding inhibits the activity of TK. This is important because TK activity can lead to bronchoconstriction, only useful during exposure to certain stimuli. Airway LPO, on the other hand, should be active at all times because it contributes to host defense against bacteria. In fact, measurements of airway LPO activity in vitro according to published methods revealed that the enzyme did not change its activity whether or not HA was present over a large concentration range.

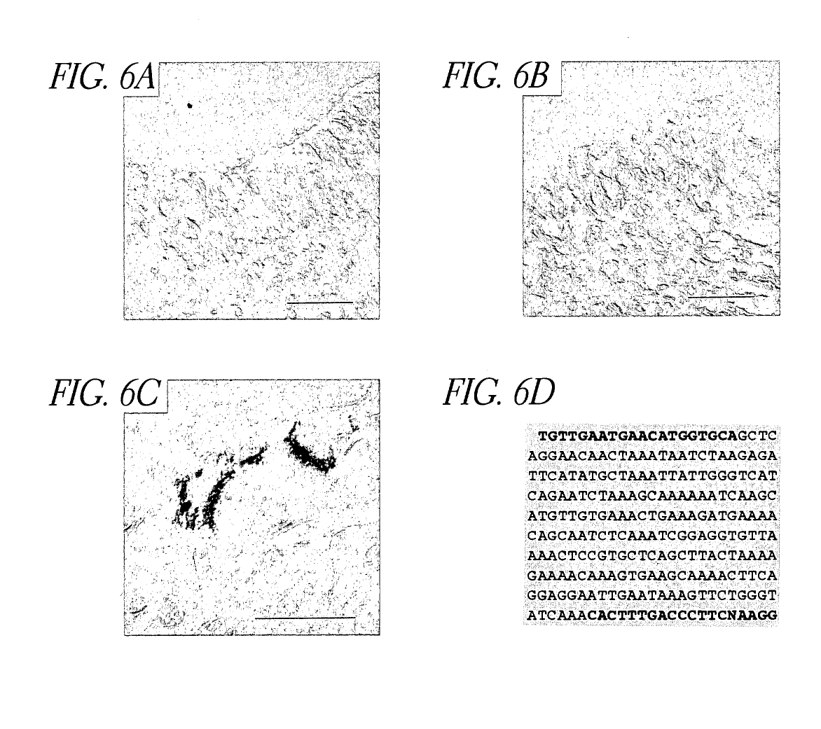

An HA-binding receptor expressed at the apical surface of the epithelium is involved in mediating the interaction between HA and TK. Previous reports indicated that CD44, a common extracellular HA receptor, is not found on the apex of normal airway epithelial cells. However, the expression of RHAMM, now also called CD168, in ovine trachea was determined using a polyclonal antibody. Immunohistochemistry revealed specific staining for RHAMM in the apical portion of ciliated cells, but no staining in goblet cells (FIG. 6). This suggests a role for RHAMM in ciliated cells.

To confirm expression of RHAMM in tracheal epithelial cells, an ovine tracheal cDNA library and primers for RHAMM were used (FIG. 6), which were designed according to consensus regions of the published sequences. An ovine mucosa cDNA library was used as a template with a specific 5′ oligonucleotide and a 3-fold degenerate 3′ primer, both designed from consensus RHAMM sequences (FIG. 6). The FailSafe™ PCR system (Epicentre Technologies, Madison, Wis.) was used with annealing at 52° C. PCR yielded a band of expected size (249 bp). The fragment was sequenced (FIG. 6) and the deduced amino-acid sequence was 91% and 81% identical to the published human and mouse RHAMM sequences, respectively. Together, these data show that RHAMM is expressed in the airway epithelium and localized to the apical portion of polarized ciliated cells.

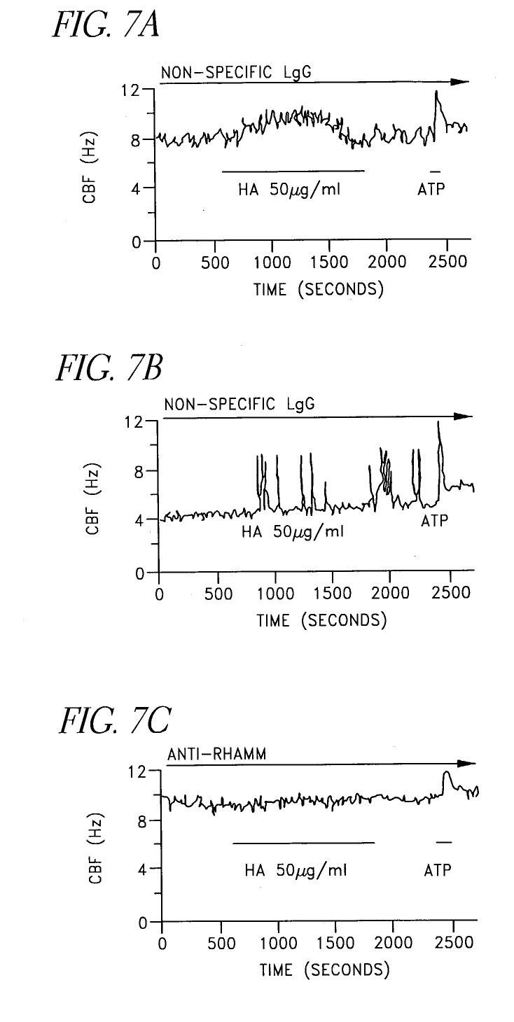

To examine whether previously reported HA-mediated increase in ciliary beat frequency (Lieb T et al. 2000 J. Aerosol Med. 231-237) was mediated by RHAMM, primary cultures of ovine airway epithelial cells were used as described (Salathe M et al. 1995 J. Cell Sci. 108:431-440). Using anti-RHAMM antibody and fixed, non-permeabilized cells, the expression of RHAMM could also be shown to occur on the surface of cultured ciliated cells (FIG. 6). These results were confirmed by adding anti-RHAMM antibody to live, cultured cells before fixation. The expression of RHAMM increased during the time in culture (18% of all ciliated cells stained positive on day 3 after plating, 57% on day 5, 65% on day 8, and 76% on day 11). This expression pattern correlated with the previously reported increase in the percentage of ciliated cells in culture staining positive for surface HA as well as the increase in the percentage of ciliated cells in culture responding to exogenous HA with an increase in ciliary beat frequency, measured by the method disclosed by Salathe M. et al. 1999 J. Physiol. (Lord.) 520:851-865. At room temperature, 6 of 8 cells more than 10 days in culture responded to 50 μg/ml HA with an increase in ciliary beat frequency from 7.2±0.6 to 9.1±0.4 Hz (p<0.05) while being exposed to a nonspecific, control rabbit anti-chicken IgG (FIG. 7). This percentage of responding cells corresponded to the percentage of RHAMM-expressing ciliated cells. On the other hand, none of 10 cells pre-incubated with a functionally blocking anti-RHAMM antibody responded with a ciliary beat frequency change (baseline 7.4±0.6 Hz; FIG. 7). Control responses to 20 μM ATP, a well-known stimulator of ciliary beat frequency, was statistically indistinguishable between both groups (ciliary beat frequency in the anti-RHAMM group was 2.5±0.5 Hz and in the anti-IgG control group 2.7±0.5 Hz, p=0.45). Since the anti-RHAMM antibody prevents HA binding to the receptor, these data show that HA-mediated changes in ciliary beat frequency occur through binding to RHAMM and further support the idea that RHAMM is an anchor for HA at the apical surface.

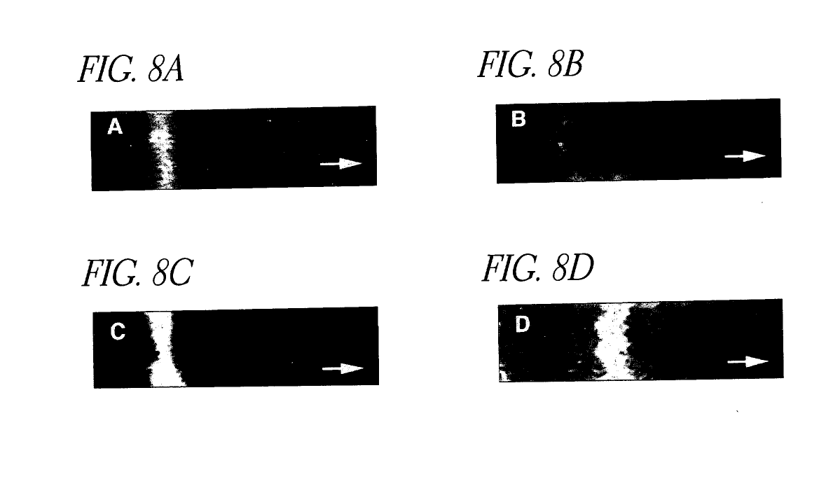

Together, these results show that HA serves a dual role in the airway epithelium by binding enzymes to the ciliary border and by simultaneously stimulating ciliary beat frequency through interactions with RHAMM. As proof of this concept, namely that HA protects these enzymes from removal by mucociliary clearance, recombinant TK was labeled with fluorescein and both airway LPO and albumin were labeled with rhodamine. Briefly, recombinant TK (gift kindly provided by Dr. Cliff Wright from Amgen Pharmaceuticals), purified airway LPO, and bovine serum albumin (Sigma), were labeled with fluorescein or rhodamine isothiocyanate according to published methods. The products were purified on Sephadex G50, concentrated to 1 mg/ml in PBS and applied in equimolar amounts to the mucosal surface of a trachea obtained from a freshly sacrificed sheep, opened by cutting through the membranous portion and kept in a humidified chamber at 37° C. The movement of the applied fluorescent substances was monitored using a broad spectrum UV-illuminator and a digital camera every 10 minutes for a total of 30 minutes. HA was removed from the surface by 5 IU/ml hyaluronidase (Worthington, active at pH 7.4). Tracheas from freshly sacrificed sheep were opened by cutting through their posterior membranous portions and kept in a humidified chamber at 37° C. First, labeled TK and albumin were applied together (as a mixture) onto the same region of the surface epithelium and the migration of the fluorescence measured over a 30 minute period. TK was not transported after application whereas albumin moved forward over the whole 30 minute period. Thus, the two substances separated which was indicated by a change of the original orange fluorescence (mixture) into a clearly defined green (TK) and red (albumin) band (FIG. 8). To show that the immobilization of TK was not due to fluorescein modification, rhodamine-labeled airway LPO was used with the same result (not shown). The immobilization of the enzymes was due to HA binding since TK and albumin did not separate on tracheas pretreated with hyaluronidase, moving at the same rate over the 30-minute period. These data show that both airway LPO and TK are bound to the airway epithelial surface by HA and are not transported away by mucociliary clearance as labeled albumin is.

In summary, HA serves a previously unrecognized pivotal role in mucosal host defense. It stimulates ciliary beating (through its interaction with RHAMM) and thereby the clearance of foreign material from mucosal surfaces, but simultaneously it retains and regulates enzymes important for homeostasis at the apical mucosal surface. Therefore, the common belief that constitutive and stimulated secretion onto the mucosal surface determines enzyme availability has to be revisited. The new paradigm shown here provides an apical enzyme pool “ready for use” and protected from ciliary clearance. It is likely that this paradigm may also apply to other mucosal surfaces such as the ones found in the mouth or gut. Thus, this apical enzyme pool will have to be considered in enzymatic reactions at the mucosal surface, be it in health or disease.

### STUDY 3

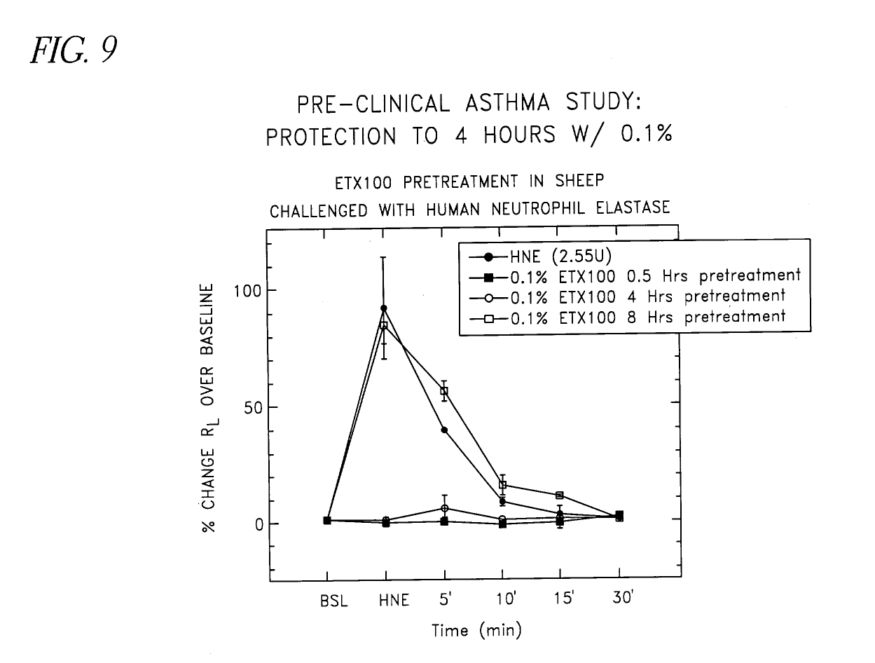

A series of experiments were conducted to demonstrate treatment and prevention of bronchoconstriction in a sheep model of asthma using aerosolized HA. The bronchoconstriction is induced by human neutrophil elastase, to mimic numerous respiratory conditions associated with neutrophil elastase release and the subsequent cascade of events that lead to increased bronchoreactivity. FIG. 9 shows the prevention of resistance in the lungs (R_{L}) (bronchoconstriction) with aerosol delivery of a formulation comprising 0.1% HA (average molecular weight of 150,000 daltons), pretreated 0.5, 4 and 8 hours before challenge with neutrophil elastase (HNE). Clearly, the HA given 0.5 and 4 hours before challenge completely ameliorated the spike in airway resistance.

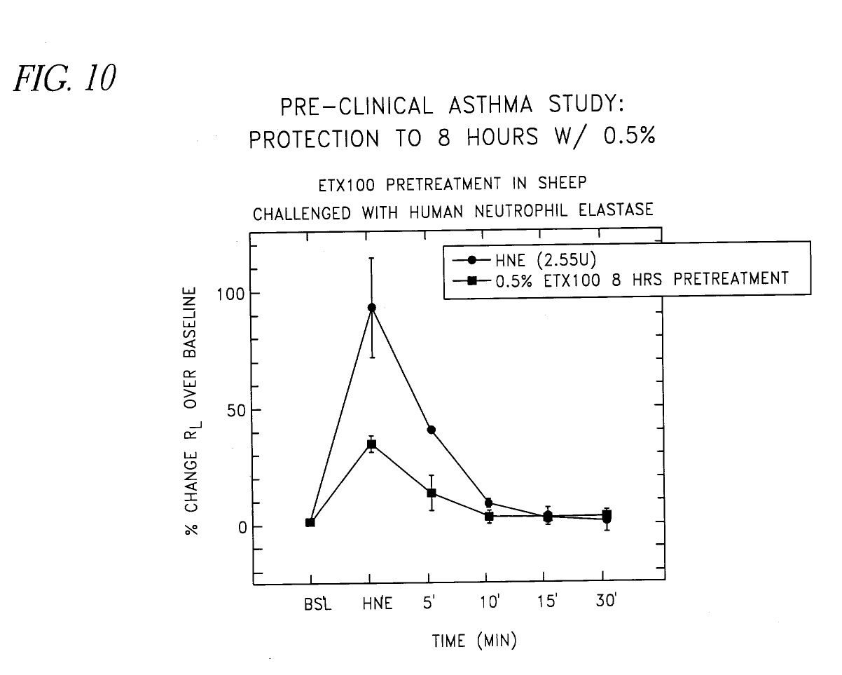

To get better prophylaxis, the dose was increased to 0.5% HA. At the higher concentration, prevention was seen even with pretreatment 8 hours before challenge with neutrophil elastase (FIG. 10).

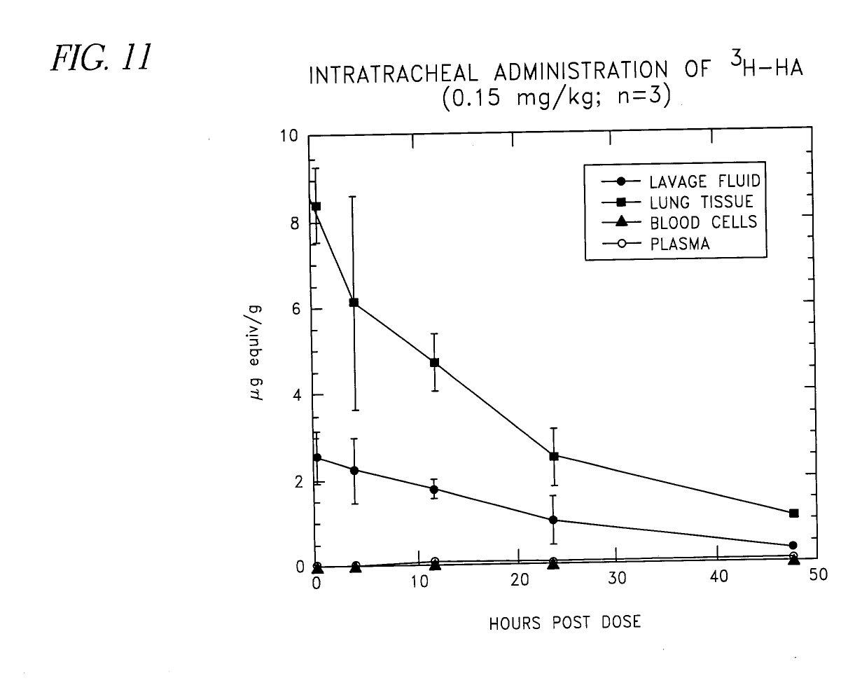

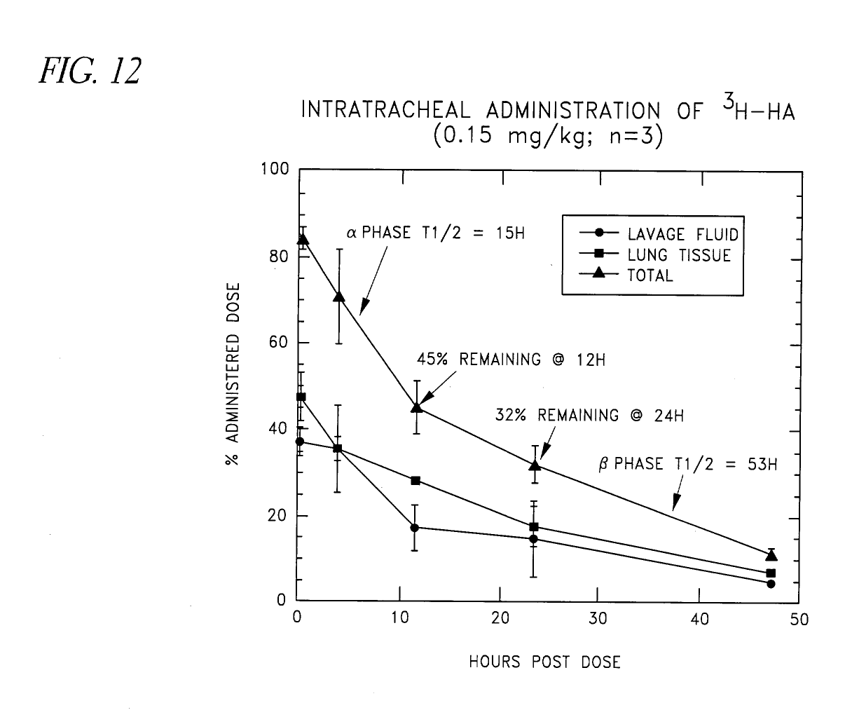

FIGS. 11 and 12 show prolonged half-life in the lungs following aerosol delivery of HA (0.15 mg/kg).

### STUDY 4

### Aerosolized HA Preparations and Characteristics

Samples solutions of HA were prepared with varying concentration for a series of different molecular weights. Molecular weights above 200,000 Dalton was measured by intrinsic viscosity and calculated by the Mark-Houwink Equation. Alternatively, molecular weight was measured by HPLC or Light Scattering analysis.

By varying the concentration for a given molecular weight of HA, a range of different viscosities were achieved. These solutions were tested in commercially available nebulizers and the mass median aerodynamic diameter (MMAD) in microns and the geometric standard deviation (GSD) were determined for each tested sample.

Samples solutions of HA were prepared. Concentrations were varied from 0.5 to 2.0 mg/ml at a molecular weight of 890,000, determined by viscometry (Table 1). A range of viscosities from 9.36 to 48.37 centistoke were achieved. These solutions were tested in Whisper, Heart and Misty nebulizers and the mass median aerodynamic diameter (MMAD) in microns and the geometric standard deviation (GSD) were determined for each tested sample. As can be seen from Table 1 below, there was a maximum limit of viscosity above which the HA solution became too viscous to nebulize. This limit is approximately 13-14 cSt for the Whisper nebulizer.

TABLE 1
Mass Median Aerodynamic
Diameter (MMAD) and Geometric Standard Deviation
(GSD) for HA Samples of about 890,000 M.W. (L-P9810-1)

| Conc. | Viscosity | | Pressure | MMAD | |
|---|---|---|---|---|---|
| mg/ml | cSt | Nebulizer | psi | (microns) | GSD |
| 2.0 | 48.37 | Whisper | 30 | TVTN* | / |
| 1.0 | 13.94 | Whisper | 30 | TVTN* | / |
| 0.5 | 9.36 | Whisper | 30 | 3.1 | 3.7 |
| 0.5 | 9.36 | Heart | 15 | 5.7 | 4.6 |
| 0.5 | 9.36 | Heart | 30 | 5.7 | 3.8 |
| 0.5 | 9.36 | Misty | 15 | 6.3 | 6.3 |
| 0.5 | 9.36 | Misty | 30 | 4.7 | 4.7 |
| 0.5 | 9.36 | Whisper | 15 | 5 | 5 |
| 0.5 | 9.36 | Whisper | 30 | 2.9 | 3.8 |

*TVTN = too viscous to nebulize

Samples solutions of HA were prepared. Concentrations were varied from 0.5 to 2.0 mg/ml at a molecular weight of 587,000, determined by viscometry (Table 2). A range of viscosities from 7.36 to 32.84 centistoke were achieved. These solutions were tested in Whisper nebulizers and the mass median aerodynamic diameter (MMAD) in microns and the geometric standard deviation (GSD) were determined for each tested sample.

TABLE 2
Mass Median Aerodynamic
Diameter (MMAD) and Geometric Standard Deviation
(GSD) for HA Samples of about 587,000 M.W. (L-9411-1)

| Conc. | Viscosity | | Pressure | MMAD | |
|---|---|---|---|---|---|
| (mg/ml) | (centistoke) | Nebulizer | (psi) | (microns) | GSD |
| 2.0 | 32.84 | Whisper | 30 | TVTN* | / |
| 1.0 | 13.56 | Whisper | 30 | 4.0 | 4.0 |
| 0.5 | 7.36 | Whisper | 30 | 6.2 | 3.8 |

*TVTN = too viscous to nebulize

Samples solutions of HA were prepared. Concentrations were varied from 0.5 to 2.0 mg/ml at a molecular weight of 375,000 as determined by HPLC (Table 3). A range of viscosities from 3.29 to 12.32 centistoke were achieved. These solutions were tested in Misty nebulizers and the mass median aerodynamic diameter (MMAD) in microns and the geometric standard deviation (GSD) were determined for each tested sample.

TABLE 3
Mass Median Aerodynamic
Diameter (MMAD) and Geometric Standard Deviation
(GSD) for HA Samples of about 375,000 M.W. (B-04m81R)

| Conc. | Viscosity | | Pressure | MMAD | |
|---|---|---|---|---|---|
| (mg/ml) | (centistoke) | Nebulizer | (psi) | (microns) | GSD |
| 2.0 | 12.32 | Misty | 15 | 5.0 | 5.4 |
| 1.0 | 5.43 | Misty | 15 | 5.2 | 6.1 |
| 0.5 | 3.29 | Misty | 15 | 6.1 | 5.8 |

Samples solutions of HA were prepared. Concentrations were varied from 0.5 to 2.0 mg/ml at a molecular weight of 350,000, determined by viscometry (Table 4). A range of viscosities from 5.56 to 7.14 centistoke were achieved. These solutions were tested in Whisper nebulizers and the mass median aerodynamic diameter (MMAD) in microns and the geometric standard deviation (GSD) were determined for each tested sample.

TABLE 4
Mass Median Aerodynamic
Diameter (MMAD) and Geometric Standard Deviation
(GSD) for HA Samples of about 350,000 M.W. (L-P9706-8)

| Conc. | Viscosity | | Pressure | MMAD | |
|---|---|---|---|---|---|
| (mg/ml) | (centistoke) | Nebulizer | (psi) | (microns) | GSD |
| 2.0 | 7.14 | Whisper | 30 | 3.0 | 3.7 |
| 1.0 | 7.09 | Whisper | 30 | 4.0 | 3.6 |
| 0.5 | 5.56 | Whisper | 30 | 3.0 | 3.2 |

Samples solutions of HA were prepared. Concentrations were varied from 0.5 to 5.0 mg/ml at a molecular weight of 220,000, determined by viscometry (Table 5). A range of viscosities from 3.60 to 6.88 centistoke were achieved. These solutions were tested in Whisper and Misty nebulizers and the mass median aerodynamic diameter (MMAD) in microns and the geometric standard deviation (GSD) were determined for each tested sample.

TABLE 5
Mass Median Aerodynamic
Diameter (MMAD) and Geometric Standard Deviation
(GSD) for HA Samples of about 220,000 M.W. (L-9711-4)

| Conc. | Viscosity | | Pressure | MMAD | |
|---|---|---|---|---|---|
| (mg/ml) | (centistoke) | Nebulizer | (psi) | (microns) | GSD |
| 2.0 | 6.88 | Whisper | 30 | 3.0 | 3.0 |
| 1.0 | 4.01 | Whisper | 30 | 4.9 | 4.5 |
| 0.5 | 3.60 | Whisper | 30 | 4.4 | 4.0 |
| 5.0 | 6.88? | Misty | 15 | 3.37 | 4.8 |
| 2.0 | 6.88 | Misty | 15 | 4.97 | 4.9 |
| 1.0 | 4.01 | Misty | 15 | 4.03 | 4.1 |
| 0.5 | 3.60 | Misty | 15 | 5.23 | 5.0 |

Samples solutions of HA were prepared. Concentrations were varied from 0.5 to 2.0 mg/ml at a molecular weight of 150,000, determined by HPLC and light scattering (Table 6). A range of viscosities from 1.72 to 3.04 centistoke were achieved. These solutions were tested in Whisper nebulizers and the mass median aerodynamic diameter (MMAD) in microns and the geometric standard deviation (GSD) were determined for each tested sample.

TABLE 6
Mass Median Aerodynamic
Diameter (MMAD) and Geometric Standard Deviation
(GSD) for HA Samples of about 150,000 M.W. (C-11097)

| Conc. | Viscosity | | Pressure | MMAD | |
|---|---|---|---|---|---|
| (mg/ml) | (centistoke) | Nebulizer | (psi) | (microns) | GSD |
| 2.0 | 3.04 | Whisper | 30 | 3.4 | 2.0 |
| 1.0 | 2.24 | Whisper | 30 | 2.1 | 2.3 |
| 0.5 | 1.72 | Whisper | 30 | 2.8 | 2.5 |

Samples solutions of HA were prepared. Concentrations were varied from 1.0 to 5.0 mg/ml at a molecular weight of 140,000, determined by HPLC (Table 7). A range of viscosities from 2.5 to 6.93 centistoke were achieved. These solutions were tested in AeroEclipse, Pari, and Misty nebulizers and the mass median aerodynamic diameter (MMAD) in microns and the geometric standard deviation (GSD) were determined for each tested sample.

TABLE 7
Mass Median Aerodynamic
Diameter (MMAD) and Geometric Standard Deviation
(GSD) for HA Samples of about 140,000 M.W. (B-173-EXP001(A & B))

| Conc. | Viscosity | | Pressure | MMAD | |
|---|---|---|---|---|---|
| (mg/ml) | (centistoke) | Nebulizer | (psi) | (microns) | GSD |
| 5.0 | 6.93 | AeroEclipse | 15 | 1.4 | 2.8 |
| 5.0 | 6.93 | AeroEclipse | 30 | 1.3 | 4.8 |
| 2.0 | 3.60 | AeroEclipse | 30 | 3.1 | 3.2 |
| 1.0 | 2.53 | AeroEclipse | 30 | 3.3 | 2.8 |
| 5.0 | 6.9 | Pari | 15 | 2.7 | 3.2 |
| 2.0 | 3.6 | Pari | 15 | 4.3 | 3.4 |
| 1.0 | 2.5 | Pari | 15 | 6.9 | 3.7 |
| 5.0 | 6.9 | Misty | 15 | 4.2 | 3.9 |
| 2.0 | 3.6 | Misty | 15 | 5.2 | 3.4 |
| 1.0 | 2.5 | Misty | 15 | 5.7 | 3.5 |

Samples solutions of HA were prepared. Concentrations were varied from 1.0 to 5.0 mg/ml at a molecular weight of 108,000, determined by light scattering (Table 8). A range of viscosities from 1.9 to 3.7 centistoke were achieved. These solutions were tested in AeroEclipse, Pari, and Misty nebulizers and the mass median aerodynamic diameter (MMAD) in microns and the geometric standard deviation (GSD) were determined for each tested sample.

TABLE 8
Mass Median Aerodynamic
Diameter (MMAD) and Geometric Standard Deviation
(GSD) for HA Samples of about 108,000 M.W. (G-9983-1B)

| Conc. | Viscosity | | Pressure | MMAD | |
|---|---|---|---|---|---|
| (mg/ml) | (centistoke) | Nebulizer | (psi) | (microns) | GSD |
| 5.0 | 3.7 | AeroEclipse | 15 | 1.9 | 2.4 |
| 5.0 | 3.7 | AeroEclipse | 30 | 2.5 | 2.9 |
| 2.0 | 2.3 | AeroEclipse | 30 | 3.3 | 2.6 |
| 1.0 | 1.9 | AeroEclipse | 30 | 3.7 | 2.3 |
| 5.0 | 3.7 | Pari | 15 | 3.5 | 3.2 |
| 2.0 | 2.3 | Pari | 15 | 6.2 | 3.8 |
| 1.0 | 1.9 | Pari | 15 | 4.2 | 3.4 |
| 5.0 | 3.7 | Misty | 15 | 3.3 | 4.0 |
| 2.0 | 2.3 | Misty | 15 | 6.0 | 3.8 |
| 1.0 | 1.9 | Misty | 15 | 4.6 | 3.7 |

The nebulizer droplet size distributions tended to be bimodal with one mode for sizes larger than about 2 μm in aerodynamic diameter and one mode smaller than about 0.5 μm. Both of these modes are relatively effectively deposited in the lung airways during inhalation and the balance between these modes determines the effective regional deposition of aerosol between the conducting airways and the deep lung These bimodal size distributions are a result of the complex interaction of evaporation phenomena for aerosols from aqueous solutions. Small droplets have higher vapor pressure than larger droplets by virtue of their surface curvature so that small droplets tend to evaporate and larger droplets grow under saturated water vapor conditions. Simultaneously, evaporation is inhibited by the HA in solutions so that the smaller droplets do not completely evaporate and may actually have a higher HA concentration per droplet volume than found in the larger droplets. The result is a bimodal distribution whose exact characteristics depends in part on the selected HA concentration.

Aerosol volumetric output concentration tends to be lower with concentrations of 5 mg/ml than for the lower concentrations (1 mg/ml and 2 mg/ml) all three nebulizers (Misty, Pari, and AeroEclipse). This does not mean that there is proportionately less HA generated at 5 mg/ml since the concentration in solution is much higher. For example, the Misty with 5 mg/ml of HA operated at 15 psig air pressure provides an aerosol of about 15.5 μl/l in 5.73 l/min. of air for a total of 15.5 μl/l×5.73 l/min.=88.8 μl/min. or 0.0888 ml/min of aerosol generated with the 5 mg/ml concentration. In comparison, at 2 mg/ml HA concentration, the aerosol output was 25.1 μl/l×5.73 l/min.=144 μl/min. or 0.144 ml/min. of aerosol. The total HA aerosolized is therefore 0.144 ml/min.×2 mg/ml=0.29 mg/min. of HA aerosol generated with the 2 mg/ml concentration. Although 5 mg/ml is 2.5 times as concentrated as 2 mg/ml, the HA output is only 1.5 more at the higher concentration. If during a twenty minute treatment period, a patient inhales for half of those twenty minutes for the aerosol generated with the 2 mg/ml solution, the inhaled HA would be 0.29 mg/min×10 min.=2.9 mg inhaled. If 60% is deposited in the lung, a total of about 1.7 mg of HA will be deposited in the lungs during this treatment.

The nebulizers acted differently in direct comparison tests. The Misty nebulizer tended to yield undesirable large geometric standard deviations in all tests. The AeroEclipse tended to give smaller droplet size standard deviations, a desirable characteristic.

The use of auxiliary air with the AeroEclipse proved highly successful. The augmentation of aerosol was ideal, with the aerosol concentration remaining about the same with and without auxiliary air. Of course, this means that the aerosol output rate was significantly increased. At a total flow rate of 18 l/min., which is equivalent to the inspiratory demand of a typical person, with 2 mg/ml HA concentration, the aerosol output during inhalation is given by 31.5 μl/l×18 l/min=567 μl/min. or 0.576 ml/min. If during a twenty minute treatment period a patient inhales for half of those twenty minutes, the inhaled HA would be 0.575 ml/min.×10 min.×2 mg/ml HA=11.3 mg inhaled. If 60% is deposited in the lung, a total of about 7 mg of HA will be deposited in the lungs during this treatment.

As previously noted, aerosol droplet size distributions with MMAD larger than 10 μm probably will result in excessive upper respiratory deposition rather than the more desirable alveolar deposition during transoral inhalation by humans. Droplet distributions in the MMAD range from 2 to 4 μm are most desirable for therapeutic studies.

Since dilution air is normally required during actual inhalation treatment, some shrinkage of droplets by evaporation may occur, and that can lead to reduced deposition. On the other hand, using a nebulizer that allows auxiliary air to pass through the nebulization zone adding aerosol to that auxiliary air can significantly increase the aerosolization rate and the deposition of HA during a given time period of inhalation treatment. The results found with AeroEclipse nebulizer demonstrate this advantageous use of auxiliary air. That auxiliary air is automatically drawn into the nebulizer from the room in response to the inhalation demand of a patient.

Further, the nebulizer and formulation must be compatible such that the process of producing a respirable aerosol affects no significant changes in HA molecular size or integrity. Examples of such formulation and nebulizer combinations are presented in Table 9.

TABLE 9
Nebulizer and Formulation Compatibility
AeroEclipse nebulizer and formulation compatibility
Nebulizer conditions
as described previously for particle size determinations.
HPLC Conditions: TSK SEC G6000 PW column (7 5 × 750 mm)
Mobile phase = 3 mM NaPO4, 0.15 M NaCl, pH 7.0, Run
time = 15 min, Injection volume = 100 uL, Detection = UV at 220 nm;
Flow rate = 1 0 mL/min

| | Pre-nebulization | Post-nebulization | |
|---|---|---|---|
| Formulation | MW (kD) | MW (kD) | % change |
| Genzyme 9983- | 96,304 | 100,990 | 4.6 |
| P-9708-4A | 387,010 | 393,911 | 1.8 |
| P9711-4 | 215,093 | 207,573 | −3.5 |
| Bayer 173 | 164,729 | 189,062 | 4.6 |

These data show less than +/−5% difference in MW resulting from the aerosolization process, and demonstrate that selection of an appropriate combination of nebulizer and formulation will ensure delivery to the patient of a controlled and specified drug product.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A method of treating or preventing respiratory conditions associated with tissue kallikrein-induced bronchoconstriction and/or airway hyperreactivity, comprising administering to a mammal in need thereof an amount of an aerosolized formulation comprising a polysaccharide capable of binding to CD44 and/or RHAMM cell surface receptors at a location along an airway epithelium, said amount being sufficient to sequester tissue kallikrein to said location, wherein an enzymatic activity of the tissue kallikrein is inhibited, thereby treating or preventing the respiratory condition.

2. The method of claim 1, wherein the polysaccharide is a glycosaminoglycan.

3. The method of claim 2, wherein the glycosaminoglycan is selected from the group consisting of hyaluronic acid, chondroitin sulfate A, chondroitin sulfate B, chondroitin sulfate C, heparan sulfate and heparin.

4. The method of claim 1, wherein the polysaccharide is hyaluronic acid.

5. The method of claim 1, wherein said administering an aerosolized formulation further comprises:
preparing a liquid formulation comprising the polysaccharide, wherein the concentration of the polysaccharide is less than about 5 mg/ml and the molecular weight of the polysaccharide is less than about 1.5×10⁶ Daltons;
aerosolizing said liquid formulation to form a breathable mist such that the particle size of the polysaccharide is less than about 10 microns; and
delivering said amount of the polysaccharide by inhalation of said breathable mist by said mammal.

6. The method of claim 5, wherein the molecular weight of the polysaccharide is less than about 587,000 Daltons.

7. The method of claim 5, wherein the molecular weight of the polysaccharide is less than about 220,000 Daltons.

8. The method of claim 5, wherein the molecular weight of the polysaccharide is less than about 150,000 Daltons.

9. The method of claim 5, wherein said breathable mist is formed by a nebulizer.

10. The method of claim 9, wherein said nebulizer operates at a pressure of at least about 15 psi.

11. The method of claim 9, wherein said nebulizer operates at a pressure of at least about 30 psi.

12. The method of claim 1, wherein the polysaccharide is chemically modified.

13. The method of claim 12, wherein the modification comprises cross-linking.

14. The method of claim 12, wherein the modification comprises addition of a functional group selected from the group consisting of sulfate group, carboxyl group, lipophilic side chain, acetyl group, and ester.

15. The method of claim 1, wherein the location is a ciliated border of the airway epithelium.

16. The method of claim 1, wherein said amount of polysaccharide is in a range of about 10 μg/kg body weight/day to about 10 mg/kg body weight/day.

17. The method of claim 1, further comprising a step of monitoring tissue kallikrein activity via bronchoalveolar lavage.

18. A method of treating or preventing respiratory conditions associated with tissue kallikrein-induced bronchoconstriction and/or airway hyperreactivity, comprising administering to a mammal in need thereof an aerosolized formulation comprising hyaluronic acid in an amount sufficient to bind to RHAMM cell surface receptors at a ciliated border of an airway epithelium and sequester tissue kallikrein to the ciliated border, thereby treating or preventing the respiratory condition.

19. A method for preventing acute bronchoconstriction due to an induction of neutrophil elastase, comprising administering to a mammal at least four hours prior to the induction an aerosolized formulation comprising hyaluronic acid at a concentration of at least 0.1% (w/v) with an average molecular weight of 150,000 daltons.

20. The method of claim 19, wherein the hyaluronic acid is administered at least eight hours prior to the induction at a concentration of at least 0.5% (w/v).
